(19) United States
(12) Patent Application Publication
(10) Pub. No.: US 2015/0004686 A1
(43) Pub. Date: Jan. 1, 2015

(54) AUTOMATIC CONTINUOUS PERFUSION CELL CULTURE MICROPLATE CONSUMABLES

(71) Applicant: CORNING INCORPORATED, Corning, NY (US)

(72) Inventors: Vasiliy Nikolaevich Goral, Painted Post, NY (US); Fang Lai, Deer Park, IL (US); Po Ki Yuen, Painted Post, NY (US)

(21) Appl. No.: 14/375,032

(22) PCT Filed: Jan. 31, 2013
(86) PCT No.: PCT/US2013/024030

§ 371 Date: Jul. 28, 2014

Related U.S. Application Data
(60) Provisional application No. 61/594,039, filed on Feb. 2, 2012.

(51) Int. Cl. C12M 1/00

(52) U.S. Cl. CPC C12M 29/10; B32B 37/12
USPC 435/305.1; 156/60; 156/257

(57) ABSTRACT

A microplate for culturing cells with automatic, continuous perfusion includes a well frame and a planar substrate. The well frame and the planar substrate form a first well, a second well, and a third well. The first well is fluidly connected with the second well with a first perfusion membrane and the first well is fluidly connected with the third well with a second perfusion membrane. Methods of fabricating the microplate and methods of culturing cells are also provided.

This application claims the benefit of priority under 35 U.S.C. §119 of U.S. Provisional Application Ser. No. 61/594,039 filed on Feb. 2, 2012 the content of which is relied upon and incorporated herein by reference in its entirety.

### BACKGROUND

1. Field

The present specification generally relates to microplates and, more specifically, to microplates for culturing cells with automatic, continuous perfusion of a liquid medium, to methods of fabricating microplates for culturing cells with automatic, continuous perfusion of a liquid medium, and to methods of culturing cells with automatic, continuous perfusion of a liquid medium.

2. Technical Background

Conventional microplates are commonly employed in laboratories as an in vitro cell culture tool. As a cell culture tool, conventional microplates provide a static cell culture microenvironment which fails to mimic in vivo microenvironments. As a result, the performance of long term cell cultures requires manual medium exchange to provide nutrients to cells and to remove waste; however, such medium exchange may result in the disturbance and removal of healthy cells from wells. While some perfusion-based cell culture systems have been developed to provide a dynamic cell culture microenvironment, current perfusion-based systems employ microfluidic devices which require the use of external pumps with tubing and/or the microfabrication of microchannels. Such systems are limited in that the formation of air bubbles in the external tubing and/or the microchannels adversely affects perfusion rates.

Accordingly, ongoing needs exist for alternative microplate designs for culturing cells with automatic, continuous perfusion of a liquid medium.

### SUMMARY

According to one embodiment, a microplate for culturing cells with automatic, continuous perfusion of a liquid medium is provided. The microplate may include a well frame which defines a plurality of cavities therethrough. The microplate may further include a planar substrate connected with the well frame. The planar substrate provides a bottom surface to the plurality of cavities, forming a plurality of wells. The plurality of wells may include a first well, a second well fluidly connected with the first well, and a third well fluidly connected with the first well. The first well may be employed for culturing the cells in the liquid medium. The second well may be employed for providing an outflow of the liquid medium to the first well. The third well may be employed for receiving an inflow of the liquid medium from the first well. The second well may be fluidly connected with the first well with a first perfusion membrane. The first perfusion membrane may be disposed in between the well frame and the planar substrate and may extend from an outlet section of the second well to an inlet section of the first well. The first perfusion membrane may have a porosity range of from about 0.2 μm to about 200 μm. The third well may be fluidly connected with the first well with a second perfusion membrane. The second perfusion membrane may be disposed in between the well frame and the planar substrate and extend from an outlet section of the first well to an inlet section of the third well. The second perfusion membrane may have a porosity range of from about 0.2 μm to about 200 μm. Upon introduction of a perfusion-initiating amount of the liquid medium into the second well, the liquid medium flows from the second well through the first perfusion membrane to the first well and from the first well through the second perfusion membrane to the third well.

In another embodiment, methods of fabricating a microplate for culturing cells with automatic, continuous perfusion of a liquid medium are provided. The methods may include providing a well frame which defines a plurality of cavities therethrough. The methods may further include positioning at least one perfusion membrane on a bottom surface of the well frame such that each of the at least one perfusion membranes extends from a first cavity of the plurality of cavities to a second cavity of the plurality of cavities, wherein the second cavity is adjacent to the first cavity. The at least one perfusion membrane has a porosity range of from about 0.2 μm to about 200 μm. The methods may further include connecting a planar substrate with the well frame, wherein the planar substrate provides a bottom surface to the plurality of cavities thereby forming a plurality of wells, and wherein the at least one perfusion membrane is disposed in between the well frame and the planar substrate.

Additional features and advantages of the embodiments described herein will be set forth in the detailed description which follows, and in part will be readily apparent to those skilled in the art from that description or recognized by practicing the embodiments described herein, including the detailed description which follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

FIG. 1 is an exploded view of a microplate for culturing cells with automatic, continuous perfusion according to embodiments described herein;

FIG. 2A is a section view of the assembled microplate taken along section line 2A-2A depicted in FIG. 1;

FIG. 2B is a section view of the assembled microplate taken along section line 2B-2B depicted in FIG. 1;

FIG. 3 is an exploded view of a microplate for culturing cells with automatic, continuous perfusion according to embodiments described herein, including the adhesive layer;

FIG. 4A is a section view of the assembled microplate taken along section line 4A-4A depicted in FIG. 3;

FIG. 4B is a section view of the assembled microplate taken along section line 4B-4B depicted in FIG. 3;

FIG. 5 is a side section view of a first well fluidly connected with a second well and a third well according to embodiments described herein;

FIG. 6A is a perspective view of a microplate for culturing cells with automatic, continuous perfusion according to embodiments described herein;

FIG. 6B is a magnified bottom view of a perfusion membrane extending from a well according to embodiments described herein;

FIG. 7 is a schematic diagram of a cross configuration according to embodiments described herein;

FIG. 8 is an exploded view of a microplate for culturing cells with automatic, continuous perfusion according to embodiments described herein, including an adhesive layer;

FIG. 9A is a section view of the assembled microplate taken along section line 9A-9A depicted in FIG. 8;

FIG. 9B is a section view of the assembled microplate taken along section line 9B-9B depicted in FIG. 8;

FIG. 10 is a graph of volume (220 μl, 120 μl, and 20 μl) of fluorescent dye solution respectively in a source (second) well, sample (first) well, and waste (third) well of a perfusion microplate with respect to fluorescent signal (unit) (A); and of time (H) with respect to volume (220 μl, 120 μl, and 20 μl) of fluorescent dye solution respectively in a source (second) well, sample (first) well, and waste (third) well of a perfusion microplate with filter paper strips with the dimensions 810 μm in width, 115 μm in thickness, and 3.5 mm in length and 320 μm in width, 115 μm in thickness, and 3.5 mm in length (B);

FIG. 11 is a live and dead staining of C3A cells (50,000/well) after 4 days of cell culture in two conventional static 96-well microplate wells without medium exchange (A), in two conventional static 96-well microplate wells with daily medium exchange (B), and two sample (first) wells of a perfusion 96-well microplate (C);

FIG. 12 is a bright field image (A)-(D) and live and dead staining (E)-(H) of EOC 20 cells (40,000/well) after 3 days of cell culture without 20% CSF-1 containing LADMAC cells cultured conditioned medium in a conventional static 96-well microplate (A), with daily 20% CSF-1 containing LADMAC-conditioned medium in a conventional static 96-well microplate (B), without 20% CSF-1 containing LADMAC conditioned medium in a perfusion 96-well microplate (C), with EMEM supplemented with 10% FBS and LADMAC cells (20,000/well) in source (second) wells of a perfusion 96-well microplate (D), without 20% CSF-1 containing LADMAC conditioned medium in a conventional static 96-well microplate (E), with daily 20% CSF-1 containing LADMAC-conditioned medium in a conventional static 96-well microplate (F), without 20% CSF-1 containing LADMAC conditioned medium in a perfusion 96-well microplate (G), and with EMEM supplemented with 10% FBS and LADMAC cells (20,000/well) in source (second) wells of a perfusion 96-well microplate (H);

FIG. 13 is a graph of time (H) of fluorescent dye solution respectively in a source (second) well, a second source (fourth) well, a sample (first) well, a waste (third) well, and a second waste (fifth) well in a cross configuration perfusion microplate with respect to fluorescent intensity (unit);

FIG. 14A is a graph of distance (m) across a sample (first) well of distribution of rhodamine dye solution in a cross configuration perfusion microplate at various time points (0 H, 0.5 H, 1 H, 1.5 H, 2 H, 2.5 H, and 3 H) with respect to normalized fluorescent intensity (unit);

FIG. 14B is a graph of distance (m) across a sample (first) well of distribution of fluorescein dye solution in a cross configuration perfusion microplate at various time points (0 H, 0.5 H, 1 H, 1.5 H, 2 H, 2.5 H, and 3 H) with respect to normalized fluorescent intensity (unit);

FIG. 15 is a live and dead staining (A)-(C) of HCT 116 colon cancer cells (17,000/well) cultured for 3 days in a sample (first) well in a perfusion microplate with human hepatocytes (50,000/well) cultured for 3 days in a source (second) well with 120 μl MFE medium in the source well (A); with 120 μl of Tegafur (40 g/ml) supplemented MFE medium in the source well (B); and with 120 μl of 5′-fluorouracil (40 g/ml) supplemented MFE medium in the source well (C);

FIG. 16 is a live and dead staining (A)-(C) of HCT 116 colon cancer cells (17,000/well) cultured for 3 days in a sample (first) well in a perfusion microplate without human hepatocytes in a source (second) well with 120 μl of 5′-fluorouracil (40 g/ml supplemented MFE in the source well (A); with 120 μl of Tegafur (40 g/ml) supplemented MFE medium in the source well (B); and with 120 μl of MFE medium supplemented (C);

FIG. 17 is a live and dead staining (A)-(C) of ReNcells (5,000/well) after 4 days of cell culture in conventional static 96-well microplate wells without medium exchange (A); in conventional static 96-well microplate wells with medium exchange every other day (B); and in sample wells of a perfusion 96-well microplate (C);

FIG. 18 is a graph of cell viability (%) of ReNcells (5,000/well) after 4 days of cell culture in conventional static 96-well microplate wells with medium exchange every other day, in conventional static 96-well microplate wells without medium exchange, and in sample wells of a perfusion 96-well microplate;

FIG. 19 is a graph of cell growth (Number of Cells) of ReNcells (5,000/well) after 4 days of cell culture in conventional static 96-well microplate wells without medium exchange, in conventional static 96-well microplate wells with medium exchange every other day, and in sample wells of a perfusion 96-well microplate;

FIG. 20 is a nestin immunostain (A)-(B) of ReNcells (5,000/well) after 4 days of cell culture in conventional static 96-well microplate wells with medium exchange every other day (A); and in sample wells of a perfusion 96-well microplate (B);

FIG. 21 is a DAPI stain (A)-(B) and brightfield image (C)-(D) of hiPSCs (20,000/well) after 4 days of culture in sample wells of a perfusion 96-well microplate (A); of hiPSCs (20,000/well) after 4 days of culture in conventional static 96-well microplate wells with daily medium exchange (B); of hESCs (20,000/well) after 4 days of culture in sample wells of a perfusion 96-well microplate (C); and of hESCs (20,000/well) after 4 days of culture in conventional static-96-well microplate wells with daily medium exchange (D);

FIG. 22 is an Oct-4 immunostain (A)-(D) of hiPSCs (20,000/well) after 4 days of culture in sample wells of a perfusion 96-well microplate (A); of hiPSCs (20,000/well) after 4 days of culture in conventional static 96-well microplate wells (B); of hESCs (20,000/well) expressing Oct-4/GFP after 4 days of culture in sample wells of a perfusion 96-well microplate (C); and of hESCs (20,000/well) after 4 days of culture in conventional static 96-well microplate wells (D);

FIG. 23 is fluorescent images of hESCs (20,000/well) expressing Oct-4/GFP (A)-(I) after 2 days of culture in conventional static 96-well microplate wells with daily medium exchange (A); after 2 days of culture in samples wells of a perfusion 96-well microplate (B); after 2 days of culture in conventional static 96-well microplate wells without daily medium exchange (C); after 3 days of culture in conventional 96-well microplate wells with daily medium exchange (D); after 3 days of culture in samples wells of a perfusion 96-well microplate (E); after 3 days of culture in conventional static 96-well microplate wells without daily medium exchange (F); after 4 days of culture in conventional 96-well microplate wells with daily medium exchange (G); after 4 days of culture in samples wells of a perfusion 96-well microplate (H); after 4 days of culture in conventional static 96-well microplate wells without daily medium exchange (I);

FIG. 23(J) is a magnified view of hESCs expressing fluorescent Oct-4/GFP shown in FIG. 23(I);

FIG. 24 a graph of cell growth (% of Colonie Area Oct4+) of hESCs (20,000/well) after 4 days of cell culture in conventional static 96-well microplate wells without daily medium exchange, in conventional static 96-well microplate wells with daily medium exchange, and in sample wells of a perfusion 96-well microplate;

FIG. 25 is a GFAP/DAPI immunostain of ReNcells (A)-(B) after 8 days of culture in conventional static 96-well microplates with medium exchange every other day (A); and after 8 days of culture in sample wells of perfusion microplates (B);

FIG. 26 is a β-III tubulin (i.e., β-III Tub) immunostain of ReNcells (A)-(B) after 8 days of culture in conventional static 96-well microplates with medium exchange every other day (A); and after 8 days of culture in sample wells of perfusion microplates (B); and

FIG. 27 is a graph of time (H) of fluorescent dye solution respectively in a source (second) well, a sample (first) well, and a waste (third) well of a perfusion microplate having perfusion membranes with varying pore sizes (1.2 μm, 5 μm, or 8 μm) with respect to volume (220 μL, 120 μL, and 20 μL) (A); and a graph of time (H) of fluorescent dye solution respectively in a source (second) well, a sample (first) well, and a waste (third) well of a perfusion microplate having perfusion membranes with varying pore sizes (1.2 μm, 5 μm, or 8 μm) with respect to volume (290 μL, 50 μL, and 20 μL) (B).

### DETAILED DESCRIPTION

Reference will now be made in detail to embodiments of a microplate for culturing cells with automatic, continuous perfusion of a liquid medium. The microplate may include a well frame which defines a plurality of cavities therethrough. The microplate may further include a planar substrate connected with the well frame. The planar substrate provides a bottom surface to the plurality of cavities, forming a plurality of wells. The plurality of wells may include a first well, a second well fluidly connected with the first well, and a third well fluidly connected with the first well. The second well may be fluidly connected with the first well with a first perfusion membrane. The first perfusion membrane may be disposed in between the well frame and the planar substrate and may extend from an outlet section of the second well to an inlet section of the first well. The third well may be fluidly connected with the first well with a second perfusion membrane. The second perfusion membrane may be disposed in between the well frame and the planar substrate and extend from an outlet section of the first well to an inlet section of the third well. Embodiments of methods of fabricating microplates for culturing cells with automatic, continuous perfusion of a liquid medium will be described in greater detail below with specific reference to the appended figures.

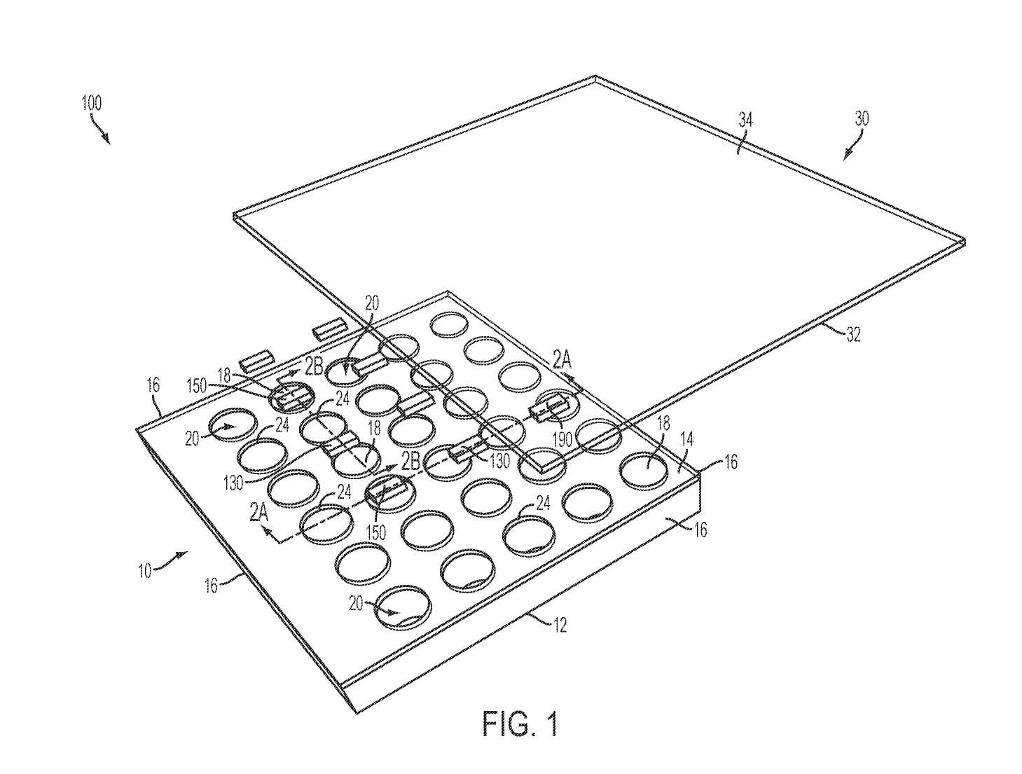

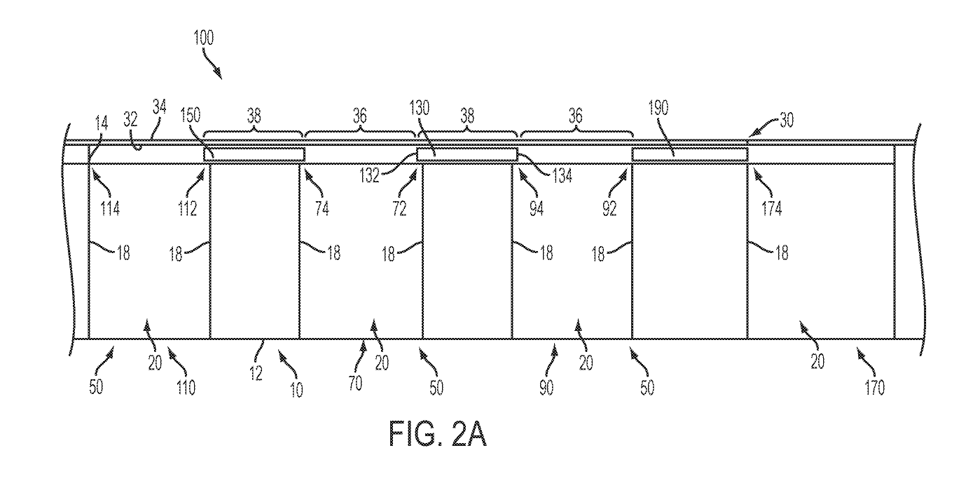

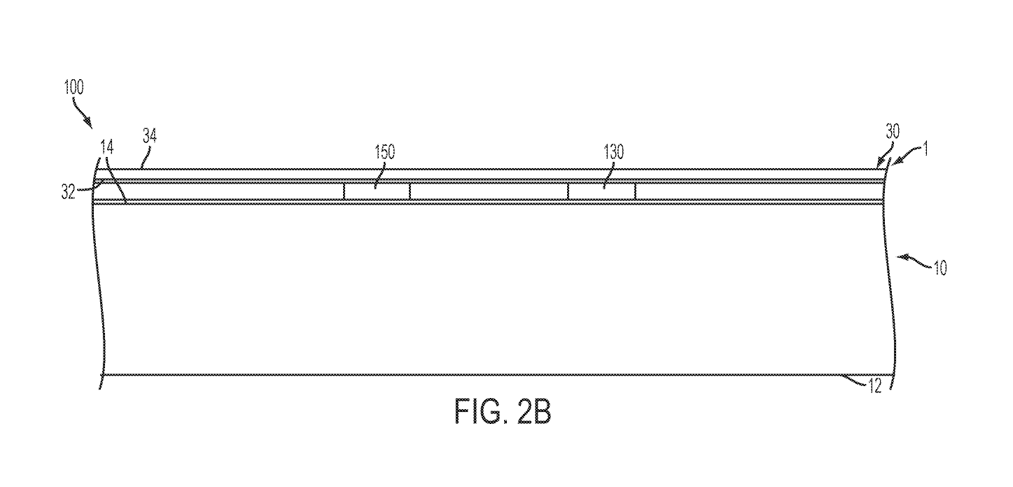

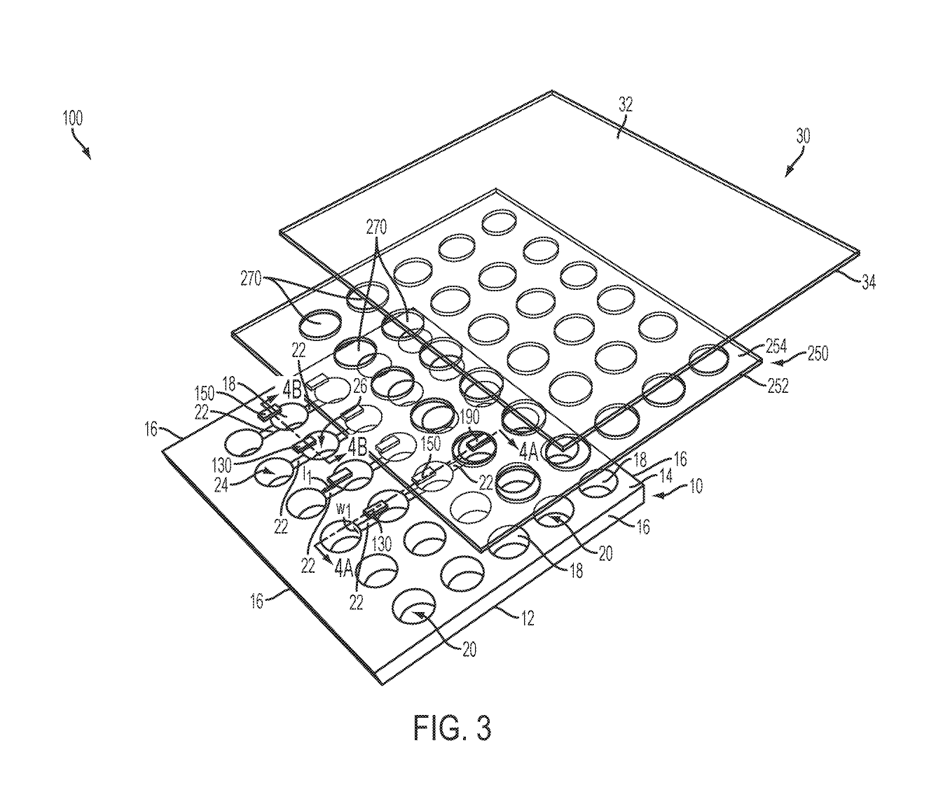

Embodiments of the well frame of the microplate will be described now with reference to FIGS. 1, 2A, and 2B. Thereafter, additional components of the microplate will be described with reference to FIGS. 3, 4A, 4B, 5-8, and 9A and 9B.

Referring to FIGS. 1, 2A, and 2B, an embodiment of the well frame 10 is depicted. The well frame 10 may include a top surface 12, a bottom surface 14, side surfaces 16, and inner surfaces 18. In some embodiments, the well frame 10 defines a plurality of cavities 20 therethrough. More specifically, the inner surfaces 18 of the well frame 10 define the cavities 20 therethrough. As used herein, the term “therethrough” refers to the extension of cavities through a structure. For example, in this embodiment, the cavities 20 extend through the well frame 10 from the top surface 12 of the well frame 10 to the bottom surface 14 of the well frame 10. In this way, each of the cavities 20 includes openings on both the top surface 12 and the bottom surface 14 of the well frame 10. In some embodiments, the cavities 20 may include, without limitation, a substantially circular or a substantially square cross-sectional shape. The cavities 20 may be arranged in rows and columns and more particularly, may be arranged in a 2:3 rectangular matrix. In some embodiments, the well frame 10 may include 6, 12, 24, 48, 96, 384 or 1536 cavities.

In some embodiments, the well frame 10 may have a substantially rectangular shape. However, further embodiments of shapes for the well frame 10 include, without limitation, circles, ovals, hexagons, pentagons, rectangles, squares, rhombuses, triangles, and even irregular shapes. The well frame 10 may be formed of materials such as, for example, polymers and/or inorganic materials. Suitable polymers may include hydrophilic polyethylene, polystyrenes, polypropylenes, acrylates, methacrylates, polycarbonates, polysulfones, polyesterketons, poly- or cyclic olefins, polychlorotrifluoroethylene, and polyethylene terephthalate. Suitable inorganic materials may include a variety of glass types such as a silicate, aluminosilicate, borosilicate, or boro-aluminosilicate, and glass ceramics, ceramics, and semiconductor or crystalline materials such as silicon. In one particular embodiment, the well frame 10 may be formed of polystyrene.

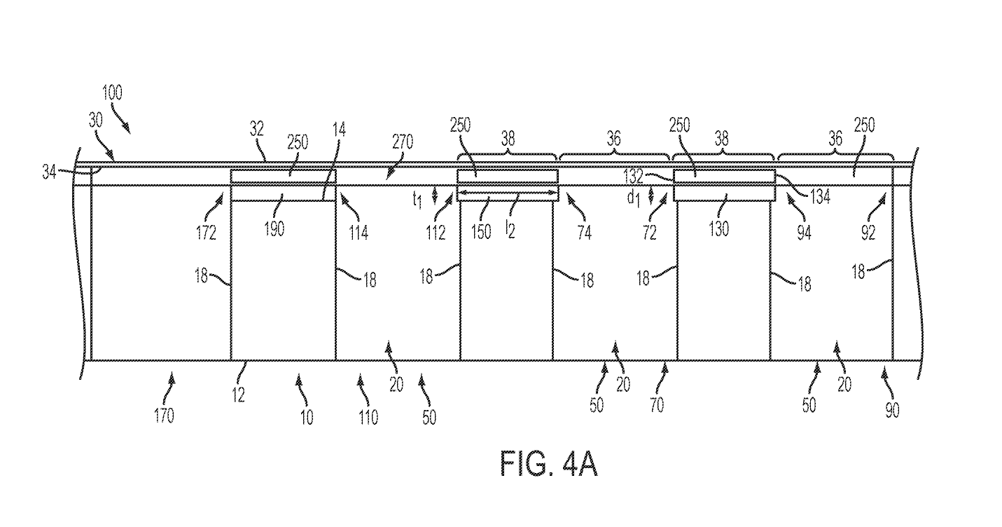

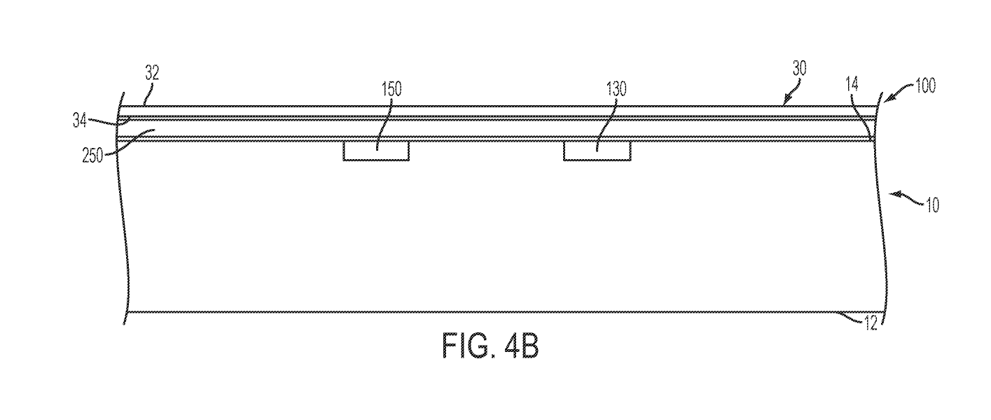

Referring now to FIGS. 3, 4A, and 4B, another embodiment of the well frame 10 is depicted. In this embodiment, the well frame 10 defines a plurality of grooves 22. More specifically, the bottom surface 14 of the well frame 10 defines the grooves 22. As will be described in greater detail below, the grooves 22 accommodate corresponding perfusion membranes. In one embodiment, the perfusion membranes may be printed in the grooves 22 by screen printing, such as described in U.S. Pat. No. 6,719,923. In one embodiment wherein the well frame 10 defines the grooves 22, the well frame 10 may be connected to a planar substrate 30 with a thermal weld, an infrared weld, or a chemical adhesive, as described in greater detail below.

More particularly, the size, shape, and positioning of the grooves 22 may correspond to the shape of the perfusion membranes. In some embodiments, the grooves 22 may have a substantially elongate shape. However, the grooves 22 may have, without limitation, any shape such that they may accommodate the corresponding perfusion membranes. In one particular embodiment, the grooves 22 have a shape which is complementary to the corresponding perfusion membranes.

In one embodiment, the grooves 22 may have a depth (as indicated by double arrow d₁) of about 50 μm to about 1000 μm, or about 100 μm to about 200 μm, or about 140 μm. The grooves 22 may also include a length (as indicated by double arrow l₁) of about 2 mm to about 55 mm, or about 3.5 mm to about 5 mm, or about 3.5 mm. The grooves 22 may also include a width (as indicated by double arrow w₁) of about 0.2 mm to about 15 mm, or about 0.5 mm to about 1.5 mm, or about 1 mm. However, it should be understood that the grooves 22 have any dimensions suitable to accommodate the corresponding perfusion membranes.

The grooves 22 are defined by the bottom surface 14 of the well frame 10 such that each groove 22 extends in between two adjacent cavities 20. More specifically, each groove 22 extends in between two adjacent cavities 20 such that when a corresponding perfusion membrane is positioned in the groove 22, the corresponding perfusion membrane extends from a first cavity 24 through the groove 22 to a second cavity 26.

Referring again to FIGS. 1, 2A, and 2B, in addition to the well frame 10, the microplate 100 also includes a planar substrate 30 connected with the well frame 10. The planar substrate 30 may be connected to the well frame 10 with a thermal weld, an infrared weld, or a chemical adhesive. The use of chemical adhesives to connect the planar substrate 30 with the well frame 10 will be described in greater detail below. The planar substrate 30 includes a top surface 32 and a bottom surface 34. In one embodiment, the top surface 32 and/or the bottom surface 34 include a plurality of first areas 36 and a plurality of second areas 38. The plurality of first areas 36 may provide a bottom surface to each of the cavities 20, thereby forming a plurality of wells 50. More specifically, upon connection of the planar substrate 30 with the well frame 10, the inner surfaces 18 of the well frame 10 and the bottom surfaces provided by the planar substrate 30 define the wells 50. Each of the wells 50 may hold from about 5 μl to about 16 ml of liquid.

In some embodiments, the planar substrate 30 may have a substantially rectangular shape. However, further embodiments of shapes for the planar substrate 30 include, without limitation, circles, ovals, hexagons, pentagons, rectangles, squares, rhombuses, triangles, and even irregular shapes. The shape of the planar substrate 30 should be such that it provides a bottom surface to each of the cavities 20. Accordingly, in some embodiments, the shape of the planar substrate 30 may match the shape of the well frame 10.

The planar substrate 30 may be formed of materials such as, for example, polymers and/or inorganic materials. Suitable polymers may include hydrophilic polyethylene, polystyrenes, polypropylenes, acrylates, methacrylates, polycarbonates, polysulfones, polyesterketons, poly- or cyclic olefins, polychlorotrifluoroethylene, and polyethylene terephthalate. Suitable inorganic materials may include a variety of glass types such as a silicate, aluminosilicate, borosilicate, or boro-aluminosilicate, and glass ceramics, ceramics, and semiconductor or crystalline materials such as silicon. In one particular embodiment, the planar substrate 30 is formed of a polystyrene film.

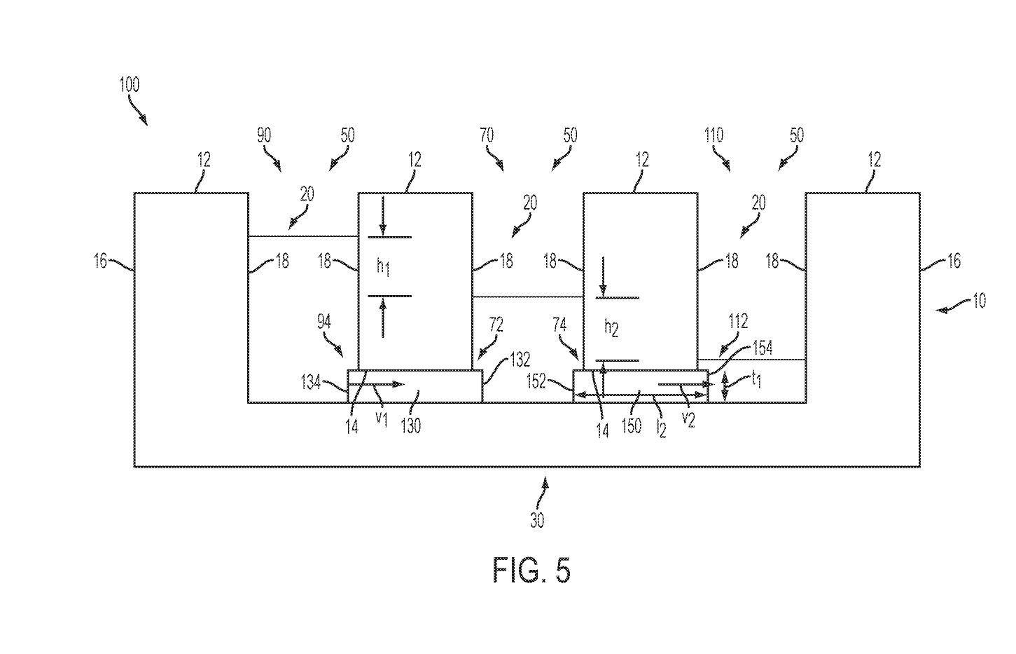

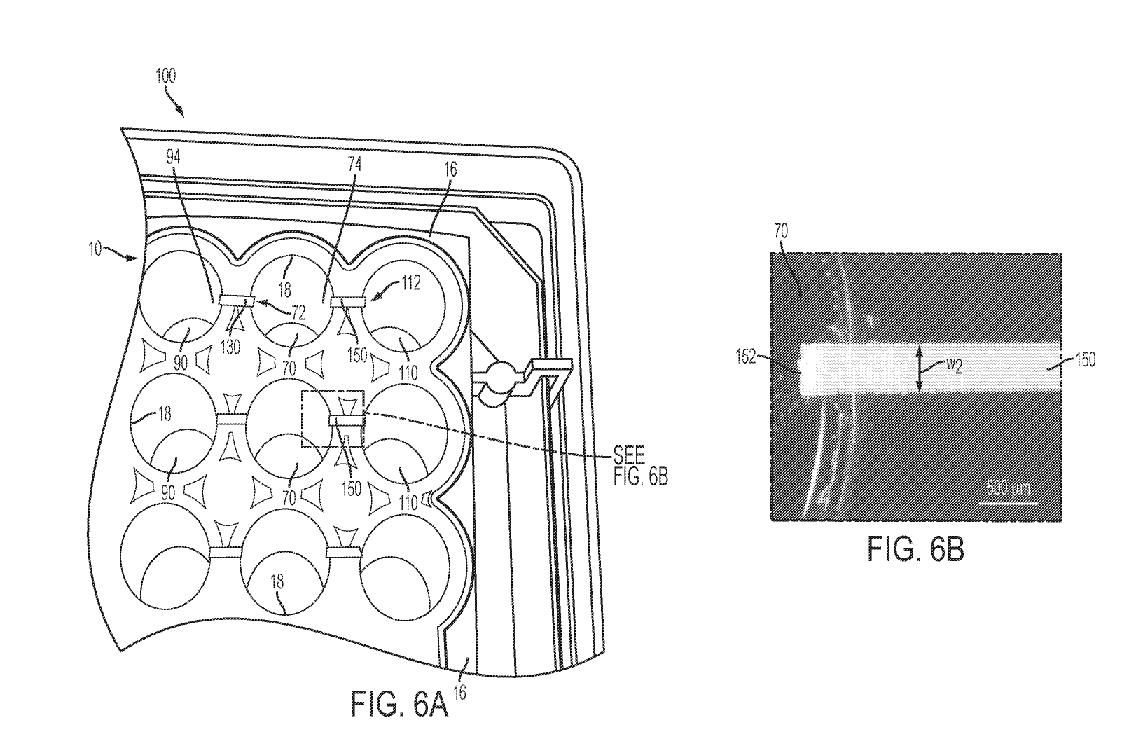

Referring now to FIGS. 5, 6A, and 6B, the plurality of wells 50 includes a first well 70, a second well 90, and a third well 110. The first well 70 is employed for culturing cells in a liquid medium. As used herein, the terms “cell culture” and “culturing cells” refers to the maintenance and/or growth of dispersed cells in a liquid medium. However, as described herein, the microplate 100 may be used for additional purposes in conjunction with culturing cells; for example, the microplate 100 may also be used for performing cell assays. The first well 70 may also be referred to as a sample well.

The second well 90 is employed for providing an outflow of a liquid medium to the first well 70. The second well 90 may also be employed for culturing cells in a liquid medium. The second well 90 is fluidly connected with the first well 70 with a first perfusion membrane 130. The second well 90 may also be referred to as a source well.

With regard to the third well 110, the third well 110 may be employed for receiving an inflow of a liquid medium from the first well 70. The third well 110 may also be employed for culturing cells in a liquid medium. The third well 110 is fluidly connected with the first well 70 with a second perfusion membrane 150. The third well 110 may also be referred to as a waste well. In this particular embodiment, the first well 70 is fluidly connected with the second well 90 and the third well 110 in an inline or series configuration.

The first perfusion membrane 130 is disposed in between the well frame 10 and the planar substrate 30. Specifically, the first perfusion membrane 130 extends from an outlet section 94 of the second well 90 to an inlet section 72 of the first well 70. In some embodiments, the first perfusion membrane 130 may extend from an outlet section 94 of the second well 90 to an inlet section 72 of the first well 70 such that a first end 132 of the first perfusion membrane extends into the cavity 20 of the first well 70 and a second end 134 of the first perfusion membrane 130 extends into the cavity 20 of the second well 90. In some embodiments, the first perfusion membrane 130 may be in direct contact with the well frame 10 and/or the planar substrate 30. In other embodiments, the first perfusion membrane 130 may be in direct contact with the well frame 10 and/or an adhesive layer which will be described in greater detail below.

The second perfusion membrane 150 may be disposed in between the well frame 10 and the planar substrate 30. More particularly, the second perfusion membrane 150 may extend from an outlet section 74 of the first well 70 to an inlet section 112 of the third well 110. In some embodiments, the second perfusion membrane 150 may extend from an outlet section 74 of the first well 70 to an inlet section 112 of the third well 110 such that a first end 152 of the second perfusion membrane 150 extends into the cavity 20 of the first well 70 and a second end 154 of the second perfusion membrane 150 extends into the cavity 20 of the third well 110. In some embodiments, the second perfusion membrane 150 may be in direct contact with the well frame 10 and/or the planar substrate 30. In other embodiments, the second perfusion membrane 150 may be in direct contact with the well frame 10 and/or an adhesive layer which will be described in greater detail below.

In some embodiments, the first perfusion membrane 130 and the second perfusion membrane 150 may have a substantially elongate shape. However, the first perfusion membrane 130 and the second perfusion membrane 150 may have, without limitation, any shape such that they may be accommodated by the corresponding grooves 22. In one particular embodiment, each of the first perfusion membrane 130 and the second perfusion membrane 150 has a shape which is complementary to the corresponding grooves 22.

Each of the first perfusion membrane 130 and the second perfusion membrane 150 may have a thickness (as indicated by double arrow t₁) of about 50 μm to about 1000 μm, or about 100 μm to about 200 μm, or about 140 μm. Each of the first perfusion membrane 130 and the second perfusion membrane 150 may also have a length (as indicated by double arrow l₂) of about 2 mm to about 55 mm, or about 3.5 mm to about 5 mm, or about 3.5 mm. Each of the first perfusion membrane 130 and the second perfusion membrane 150 may also include a width (as indicated by double arrow w₂) of about 0.2 mm to about 15 mm, or about 0.3 mm to about 1.5 mm, or about 1 mm. In one embodiment, the perfusion membranes have a porosity range of from about 0.2 μm to about 200 μm, or from about 0.2 μm to about 50 μm. In another particular embodiment, the perfusion membranes have a pore diameter of from about 1 mm to about 10 mm.

In another embodiment, each of the first perfusion membrane 130 and the second perfusion membrane 150 may be formed of a hydrophilic material. Suitable hydrophilic materials include, for example, cellulose filter papers and polymers including celluloses, nylons, polyethersulfones, and polyamides. Suitable polymers of cellulose include cellulose-derived polymers such as acetate, cellulose nitrate, and mixed cellulose esters. In one particular embodiment, the first perfusion membrane 130 and the second perfusion membrane 150 are formed of cellulose-derived polymers, and in particular, of cellulose acetate.

In one embodiment, as depicted in FIG. 5, upon introduction of a perfusion-initiating amount of the liquid medium into the second well 90, the liquid medium flows (as shown by the arrows labeled v₁ and v₂) from the second well 90 through the first perfusion membrane 130 to the first well 70 and from the first well 70 through the second perfusion membrane 150 to the third well 110.

As used herein, the term “perfusion-initiating amount” refers to the volume of liquid medium required to initiate automatic, continuous perfusion of the liquid medium. The volume of liquid medium required to initiate automatic, continuous perfusion of the liquid medium is any amount of liquid which establishes a non-equilibrium state between fluidly connected wells 50. A non-equilibrium state between two fluidly connected wells 50 is established when there is a volume difference between the two fluidly connected wells 50. Moreover, when two fluidly connected wells 50 have the same dimensions, a non-equilibrium state is established when there is a difference between the heights of the surfaces of liquid medium in the two fluidly connected wells 50. For example, a non-equilibrium state between the second well 90 and the first well 70 is established when there is a difference (as shown by arrows h₁) in the height of the surface of the liquid medium in the second well 90 and the height of the surface of the liquid medium in the first well 70. Similarly, a non-equilibrium state between the first well 70 and the third well 110 is created when there is a difference (as shown by arrows h₂) in the height of the surface of the liquid medium in the first well 70 and the height of the surface of the liquid medium in the third well 110. In some embodiments, the perfusion-initiating amount is from about 10 μl to about 16 ml, or from about 90 μl to about 290 μl, or about 220 μl of liquid medium. As used herein, the term “automatic” refers to a microplate in which perfusion of a liquid medium is accomplished without the use of external pumps.

The rate of perfusion of the liquid medium may be controlled by the dimensions of the perfusion membranes 130, 150, by the difference in volume between the first well 70, second well 90 and/or the third well 110, and/or by the difference (h₁, h₂) in heights of the liquid medium in the second well 90 fluidly connected with the first well 70 and the first well 70 fluidly connected with the third well 110. The rate of perfusion of the liquid medium may also be controlled by the porosity (i.e., pore size) of the perfusion membranes 130, 150.

In some embodiments, perfusion between two fluidly connected wells 50 continues until an equilibrium state is established between the wells 50. An equilibrium state is established when there is no volume difference between two fluidly connected wells 50. Moreover, when two fluidly connected wells 50 have the same dimensions, an equilibrium state is created when there is no difference between the heights of the surfaces of liquid medium in the two fluidly connected wells 50.

In additional embodiments, the plurality of wells 50 may include multiple series of a first well 70 fluidly connected with a second well 90 and a third well 110 as previously described. For example, in a microplate comprising 96 wells, the plurality of wells 50 may include 32 series of a first well 70 fluidly connected with a second well 90 and a third well 110 as previously described.

Referring to FIGS. 1, 2A, 3, 4A, and 7, in addition to the first well 70, the second well 90, and the third well 110, the plurality of wells 50 may further include a fourth well 170. In one embodiment, the fourth well 170 may be employed for providing an outflow of a liquid medium to the second well 90. In this particular embodiment, the fourth well 170 may be referred to as a second source well. The fourth well 170 is fluidly connected with the second well 90 with a third perfusion membrane 190. The third perfusion membrane 190 is similar to the first perfusion membrane 130 and the second perfusion membrane 150 except that the third perfusion membrane 190 extends from an outlet section 174 of the fourth well 170 to an inlet section 92 of the second well 90. In this embodiment, the first well 70 may be fluidly connected with the second well 90, the third well 110, and the fourth well 170 in an inline or series configuration.

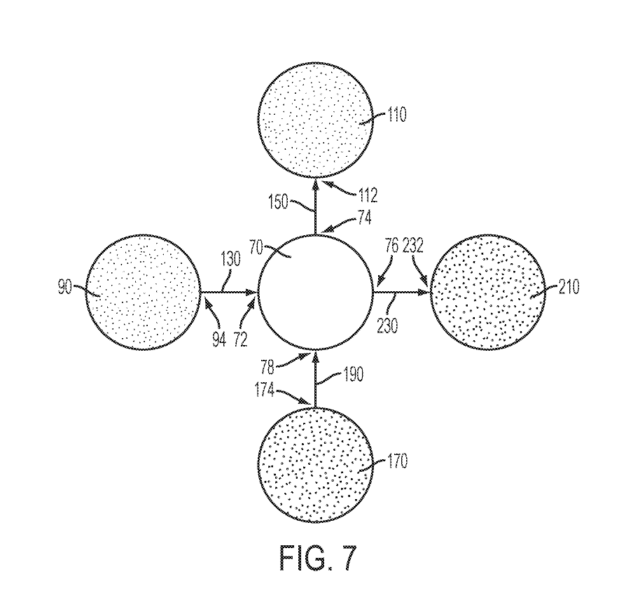

Referring particularly to FIG. 7, in a further embodiment, in addition to the fourth well 170 for providing a second outflow of the liquid medium, the plurality of wells 50 may further include a fifth well 210. In this particular embodiment, the fourth well 170 is fluidly connected with the first well 70 with a third perfusion membrane 190. The third perfusion membrane 190 extends from an outlet section 174 of the fourth well to a second inlet section 78 of the first well 70. Also in this embodiment, the fifth well 210 may be employed for receiving a second inflow of a liquid medium from the first well 70. In this particular embodiment, the fifth well 210 may be referred to as a second waste well. The fifth well 210 is fluidly connected with the first well 70 with a fourth perfusion membrane 230. The fourth perfusion membrane 230 is similar to the first perfusion membrane 130 and the second perfusion membrane 150 except that the fourth perfusion membrane 230 extends from a second outlet section 76 of the first well 70 to an inlet section 232 of the fifth well 210. In this embodiment, the first well 70 may be fluidly connected with the second well 90, the third well 110, the fourth well 170, and the fifth well 210 in a cross configuration. As used herein, the term “cross configuration” refers to two series of intersecting wells, wherein one series of wells is in a column and another series of wells is in a row. The fourth well 170 and the fifth well 210 may each be employed for culturing cells in a liquid medium.

Referring particularly to FIGS. 3 and 4A, in another embodiment, the fourth well 170 may be employed for receiving an outflow of a liquid medium from the third well 110. In this particular embodiment, the fourth well 170 may be referred to as a second waste well. The fourth well 170 is fluidly connected with the third well 110 with a third perfusion membrane 190. The third perfusion membrane 190 is similar to the first perfusion membrane 130 and the second perfusion membrane 150 except that the third perfusion membrane 190 extends from an outlet section 114 of the third well 110 to an inlet section 172 of the fourth well 170. In this embodiment, the first well 70 may be fluidly connected with the second well 90, the third well 110, and the fourth well 170 in an inline configuration.

In additional embodiments, the plurality of wells 50 may include multiple series of a first well 70 fluidly connected with a second well 90, a third well 110, a fourth well 170, and/or a fifth well 210 as previously described. For example, in a microplate comprising 96 wells, the plurality of wells 50 may include 24 series of a first well 70 fluidly connected with a second well 90, a third well 110, and a fourth well 170 as described.

Referring to FIGS. 3, 4A, 4B, 8, 9A and 9B, in addition to the well frame 10 and the planar substrate 30, in some embodiments, the microplate 100 may also include an adhesive layer 250. The adhesive layer 250 may be disposed in between the well frame 10 and the planar substrate 30. In this particular embodiment, the adhesive layer 250 connects the well frame 10 with the planar substrate 30. The adhesive layer includes a top surface 252 and a bottom surface 254. The adhesive layer 250 defines a plurality of cavities 270 therethrough. In some embodiments, the cavities 270 may have, without limitation, a substantially circular or a substantially square cross-sectional shape. The cavities 270 may be arranged in rows and columns and more particularly, may be arranged in a 2:3 rectangular matrix. The adhesive layer may include 6, 12, 24, 48, 96, 384 or 1536 cavities 270 as described above with respect to the well frame 10. In one embodiment, the cavities 270 defined by the adhesive layer 250 may correspond to the cavities 20 defined by the well frame 10. More particularly, the shape, size, and positioning of the cavities 270 defined by the adhesive layer 250 generally corresponds to the shape, size, and positioning of the cavities 20 defined by the well frame 10. In this way, the planar substrate 30 forms bottom surfaces of the cavities 20.

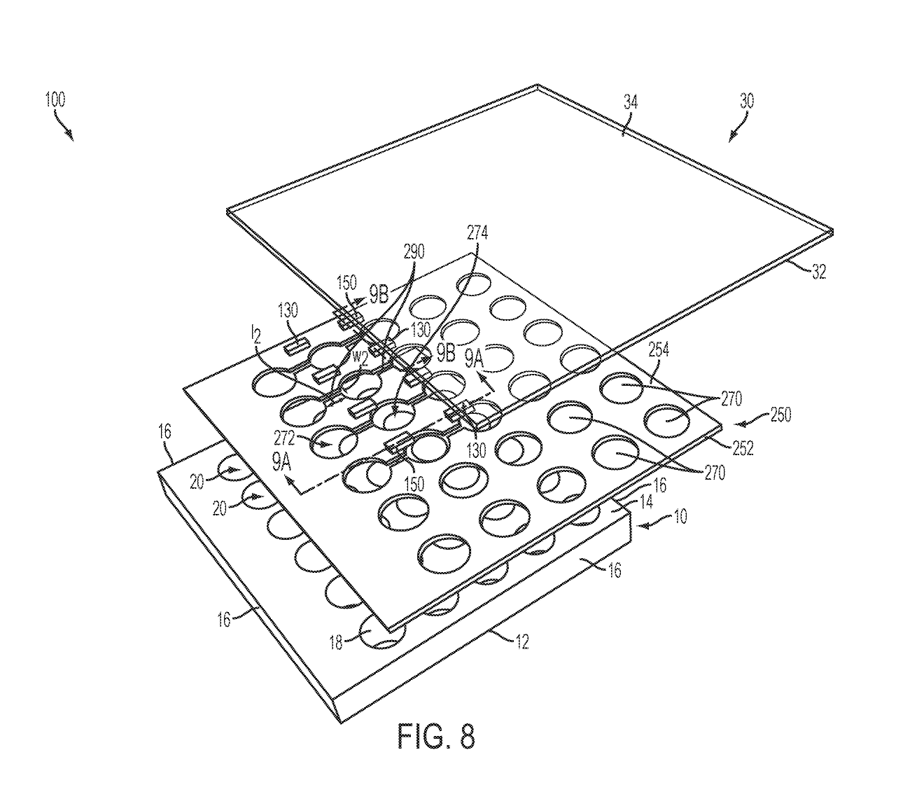

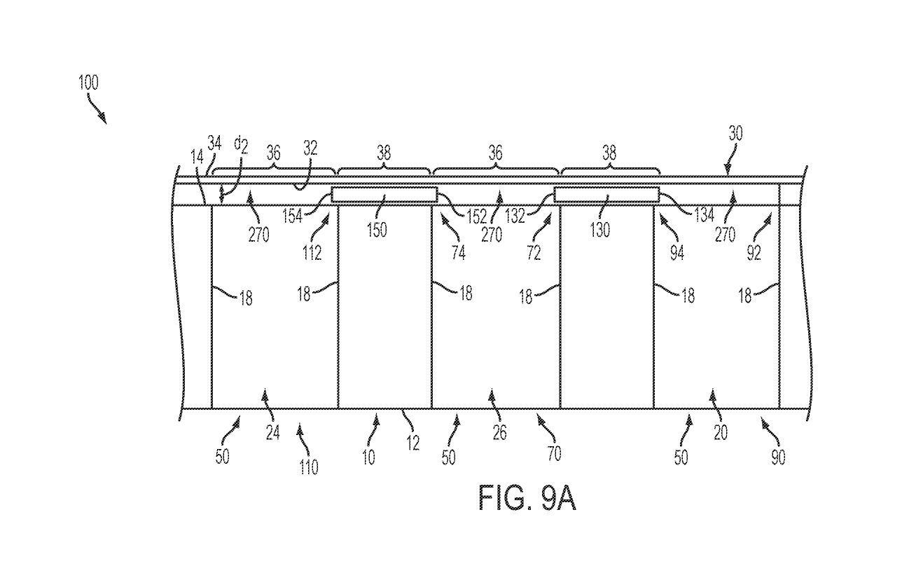

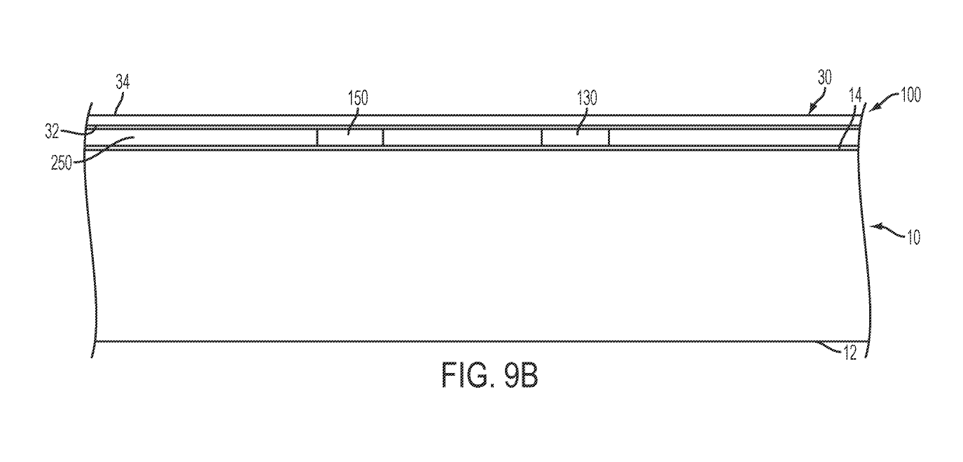

Referring particularly to FIGS. 8, 9A, and 9B, in one embodiment, the adhesive layer 250 has a plurality of channels 290 therethrough. The channels 290 are shaped to accommodate corresponding perfusion membranes. More particularly, the size, shape, and positioning of the channels 290 corresponds to the perfusion membranes. For example, in one embodiment, the channels 290 provide spaces for accommodating a corresponding first perfusion membrane 130 and a second perfusion membrane 150. In some embodiments, the channels 290 may have a substantially elongate shape. However, the channels 290 may have any suitable shape to accommodate corresponding perfusion membranes. In one particular embodiment, the channels 290 have a shape which is complementary to the corresponding perfusion membranes.

In another embodiment, the channels 290 may include a depth (as indicated by double arrow d₂) of about 50 μm to about 1000 μm, or about 100 μm to about 200 μm, or about 140 μm. The channels 290 may also include a length (as indicated by double arrow l₂) of about 2 mm to about 55 mm, or about 5 mm to about 10 mm, or about 3.5 mm. The channels 290 may also include a width (as indicated by double arrow w₂) of about 0.2 mm to about 15 mm, or about 0.5 mm to about 1.5 mm, or about 1 mm. However, it should be understood that the channels 290 may have any dimensions such that they may accommodate the corresponding perfusion membranes.

Each of the channels 290 extends between two adjacent cavities 270. More specifically, each channel 290 extends between two adjacent cavities 270 such that when a corresponding perfusion membrane is positioned in the channel, the corresponding perfusion membrane extends from a first cavity 24 in one well through the channel 290 to a second cavity 26 in a second well.

Embodiments of the microplate 100 for culturing cells with automatic, continuous perfusion of a liquid medium have been described in detail. Further embodiments directed to methods of fabricating a microplate 100 for culturing cells with automatic, continuous perfusion of a liquid medium according to one or more such embodiments will now be described.

Referring to FIGS. 1, 3, and 8, which show perspective views of a microplate 100, the methods of fabricating a microplate 100 for culturing cells with automatic, continuous perfusion of a liquid medium result in the formation of a microplate 100 as previously described. The embodiments described below of methods of fabricating a microplate for culturing cells with automatic, continuous perfusion of a liquid medium, unless noted otherwise, reference components of the microplate 100 shown in FIGS. 1, 3, and 8.

In some embodiments, methods of fabricating a microplate 100 for culturing cells with automatic, continuous perfusion of a liquid medium are provided. The methods include providing a well frame 10 which defines a plurality of cavities 20 therethrough. The methods further include positioning at least one perfusion membrane 130, 150, 190, 230 on a bottom surface 14 of the well frame 10 such that each of the at least one perfusion membranes 130, 150, 190, 230 extends from a first cavity 24 of the plurality of cavities to a second cavity 26 of the plurality of cavities, wherein the second cavity 26 is adjacent to the first cavity 24. The at least one perfusion membrane 130, 150, 190, 230 has a porosity range of from about 0.2 μm to about 200 μm. The methods may further include connecting a planar substrate 30 with the well frame 10, wherein the planar substrate 30 provides a bottom surface to the plurality of cavities 20 thereby forming a plurality of wells 50, and wherein the at least one perfusion membrane 130, 150, 190, 230 is disposed in between the well frame 10 and the planar substrate 30.

In embodiments, the methods of fabricating a microplate 100 for culturing cells with automatic, continuous perfusion of a liquid medium may include providing a well frame 10 which defines a plurality of cavities 20 therethrough.

Thereafter, at least one perfusion membrane 130, 150, 190, 230 is positioned on a bottom surface 14 of the well frame 10 such that each of the at least one perfusion membrane 130, 150, 190, 230 extends from a first cavity 24 of the plurality of cavities to a second cavity 26 of the plurality of cavities. A planar substrate 30 is then connected with the well frame 10. The planar substrate 30 provides a bottom surface to the plurality of cavities 20 thereby forming a plurality of wells 50. The at least one perfusion membrane 130, 150, 190, 230 is disposed in between the well frame 10 and the planar substrate 30.

With regard to connecting the planar substrate 30 with the well frame 10, in one embodiment, the planar substrate 30 may be connected with the well frame 10 with a pressure sensitive adhesive. Referring to FIGS. 1 and 2A, the planar substrate 30 is connected with the well frame 10 by applying the pressure sensitive adhesive to the bottom surface 14 of the well frame 10 which is applied to the bottom surface 14 of the well frame 10 prior to positioning the perfusion membrane on the bottom surface 14. In this particular embodiment, the perfusion membrane 130, 150 and the planar substrate 30 adhere to the bottom surface 14 of the well frame 10. In a further embodiment, the planar substrate 30 may include a plurality of second areas 38 which adhere to the bottom surface 14 of the well frame 10. Alternatively, in another embodiment, the planar substrate 30 may be connected with the well frame 10 with a thermal weld and/or an infrared weld.

Referring to FIG. 3, in another embodiment, the methods of fabricating a microplate 100 for culturing cells with automatic, continuous perfusion of a liquid medium may also include forming a plurality of grooves 22 in the well frame 10. The grooves 22 are formed in the well frame 10 prior to positioning the at least one perfusion membrane on the bottom surface 14 of the well frame 10. The grooves 22 extend from a first cavity 24 of the plurality of cavities to a second cavity 26 of the plurality of cavities. The grooves 22 correspond with the at least one perfusion membrane 130, 150, 190, 230 such that when positioning the perfusion membranes 130, 150, 190, 230 on the bottom surface 14 of the well frame 10, each of the perfusion membranes 130, 150, 190, 230 is positioned in a groove 22.

In a further embodiment, the pressure sensitive adhesive is an adhesive layer 250 which is applied to the bottom surface 14 of the well frame 10. The adhesive layer 250 defines a plurality of cavities 270 therethrough. The plurality of cavities 270 defined by the adhesive layer 250 corresponds with the plurality of cavities 20 defined by the well frame 10, such that the planar substrate 30 adheres to the adhesive layer 250, the adhesive layer 250 adheres to the well frame 10, and the perfusion membranes 130, 150, 190, 230 are positioned in between the well frame 10 and the adhesive layer 250.

Referring to FIG. 8, in a further embodiment, the adhesive layer 250 may define a plurality of channels 290. The channels 290 may extend from a first cavity 272 of the plurality of cavities 270 to a second cavity 274 of the plurality of cavities 270. The channels 290 correspond with the at least one perfusion membrane 130, 150, 190, 230 such that when positioning the perfusion membranes 130, 150 on the bottom surface 14 of the well frame 10, the perfusion membranes 130, 150 are positioned within the channels 290. The plurality of cavities 270 defined by the adhesive layer 250 may also correspond with the plurality of cavities 20 defined by the well frame 10, such that the planar substrate 30 adheres to the adhesive layer 250 and the perfusion membranes 130, 150, 190, 230 are positioned in between the well frame 10 and the planar substrate 30.

Embodiments of methods of fabricating a microplate 100 for culturing cells with automatic, continuous perfusion of a liquid medium have been described in detail. Further embodiments directed to methods of culturing cells according to one or more such embodiments will now be described. The embodiments described below of methods of culturing cells with automatic, continuous perfusion of a liquid medium, unless noted otherwise, reference components of the microplate 100 shown in FIGS. 1, 3, and 8.

In some embodiments, methods of culturing cells with automatic, continuous perfusion of a liquid medium are provided. The methods include providing a microplate 100, placing the cells to be cultured into at least one of the first well 70, the second well 90, and the third well 110 of the microplate 100, and culturing the cells by placing a perfusion-initiating amount of the liquid medium into the second well 90 such that the liquid medium flows from the second well 90 through the first perfusion membrane 130 to the first well 70 and from the first well 70 through the second perfusion membrane 150 to the third well 110, contacting the cells. The microplate 100 and the perfusion initiating amount of the liquid medium are as previously described.

In embodiments, the cells to be cultured are placed into the first well 70. The cells to be cultured may also be placed into the first well 70 and the second well 90. The cells to be cultured may also be placed into the first well 70 and the third well 110. Moreover, the cells to be cultured may be placed into the first well 70, the second well 90, and the third well 110.

The cells to be cultured may be cultured in free suspensions, encapsulated in suitable hydrogels, encapsulated in matrices, and/or encapsulated in scaffolds. Any cells of interest may be cultured. For example, the cells to be cultured may include animal, plant, fungi, microbe, virus, bacteria, and/or protist cells. Moreover, the cells to be cultured may include normal, mutant, stem, cancerous, and/or diseased cells. Additionally, one or more cell types may be cultured at the same time in the microplate 100. For example, cells of the same type may be cultured at the same time in different fluidly connected wells 50 of the microplate 100. Alternatively, as another example, cells of different types may be cultured at the same time in different fluidly connected wells 50 of the microplate 100. For example, a first cell type may be cultured in the first well 70 while a second cell type may be simultaneously cultured in the second well 90.

In one embodiment, the methods of culturing cells further include performing an analysis of cell-cell communication between cells cultured in the first well 70 and cells cultured in the second well 90, thereby determining an in vitro effect of the cell-cell communication on the cells cultured in the first well 70. The methods are further described in greater detail below. Briefly, the analysis may include placing a perfusion initiating amount of a liquid medium into the second well 90 such that the liquid medium contacts the cells cultured in the second well 90, flows from the second well 90 through the first perfusion membrane 130 to the first well 70, contacts the cells cultured in the first well 70, and flows from the first well 70 through the second perfusion membrane 150 to the third well 110, and determining cell viability of the cells cultured in the first well. In one embodiment, the cells provided in the second well 90 may provide molecules and/or compositions necessary for growth of the cells provided in the first well 70. For example, the cells provided in the second well 90 may provide a growth factor required for growth of the cells provided in the first well 70.

The cells cultured in the first well 70 may be the same type as or may be a different type from the cells provided in the second well 90. In one particular embodiment, the cells cultured in the first well 70 include a first cell type and the cells cultured in the second well 90 include a second cell type. Additionally, the cells may be cultured in the first well 70 and in the third well 110 such that an in vitro effect of cell-cell communication of the cells cultured in the first well 70 on the cells cultured in the third well 110 may be determined. Alternatively, the cells may be cultured in the second well 90 and in the third well 110 such that an in vitro effect of cell-cell communication of the cells cultured in the second well 90 on the cells cultured in the third well 110 may be determined. As another alternative, the cells may be cultured in the first well 70, in the second well 90, and in the third well 110 such that an in vitro effect of the cell-cell communication of the cells cultured in the first well and/or the cells cultured in the second well 90 on the cells cultured in the first well 70 and/or the third well 110 may be determined.

Cell viability, morphology, and/or growth may be determined for the cells cultured in the first well 70, second well 90, and/or the third well 110. Cell viability may be determined via viability assays and/or via live and dead staining as described in greater detail below. Cell morphology may be determined as described in greater detail below. Cell growth may be determined as described in greater detail below.

In another embodiment, the methods of culturing cells further include performing an analysis of an effect of an agent of interest on the cultured cells, thereby determining an in vitro effect of the agent of interest on the cultured cells. The methods are further described in greater detail below. Briefly, the analysis may include placing a perfusion initiating amount of a liquid medium containing the agent of interest into the second well 90 such that the agent of interest flows from the second well 90 through the first perfusion membrane 130 to the first well 70 and from the first well 70 through the second perfusion membrane 150 to the third well 110, contacting the cultured cells, and determining cell viability of the cultured cells.

In one particular embodiment, cells are cultured in the first well 70 and in the second well 90 such that the in vitro effect of the agent of interest on the cells cultured in the first well 70 may be determined. In this embodiment, the analysis includes placing a perfusion initiating amount of a liquid medium containing the agent of interest into the second well 90 such that the liquid medium contacts the cells cultured in the second well 90, flows from the second well 90 through the first perfusion membrane 130 to the first well 70, contacts the cells cultured in the first well 70, and flows from the first well 70 through the second perfusion membrane 150 to the third well 110, and determining cell viability of the cells cultured in the first well 70. The cells cultured in the first well 70 may be the same type as or may be a different type from the cells cultured in the second well 90. In one particular embodiment, the cells cultured in the first well 70 include a first cell type and the cells cultured in the second well 90 include a second cell type.

Additionally, the cells may be cultured in the first well 70 and in the third well 110 such that an in vitro effect of the agent of interest on the cells cultured in the third well 110 may be determined. Alternatively, the cells may be cultured in the second well 90 and in the third well 110 such that an in vitro effect of the agent of interest on the cells cultured in the third well 110 may be determined. As another alternative, the cells may be cultured in the first well 70, in the second well 90, and in the third well 110 such that an in vitro effect of the agent of interest on the cells cultured in the first well 70 and/or the third well 110 may be determined.

The agent of interest may be a drug, a chemical composition, and/or a toxin. In one particular embodiment, the agent of interest may be a prodrug. In one particular embodiment, the second well 90 may include cells capable of metabolizing the prodrug. In this embodiment, a prodrug may be placed in an inactive form in the second well 90 which includes cells capable of metabolizing the prodrug such that the prodrug may be metabolized to an active form by the cells in the second well 90. Accordingly, the effect of the active form of the drug on cells cultured in the first well 70 and/or the third well 110 may be determined. In an alternative embodiment, the first well 70 may include cells capable of metabolizing the prodrug and the third well 110 may include additional cultured cells. In this embodiment, a prodrug may be placed in an inactive form in the first well 70 such that the prodrug may be metabolized to an active form by the cells in the first well 70. Accordingly, the effect of the active form of the drug on cells cultured in the third well 110 may be determined.

Cell viability of the cells cultured in the first well 70, second well 90, and/or the third well 110 may be determined as previously described.

In another embodiment, the methods of culturing cells further include controlling differentiation of stem cells. In one particular embodiment, the method of culturing cells includes controlling differentiation of neural progenitor stem cells. In a further embodiment, the method of culturing cells includes controlling differential of neural progenitor stem cells to astrocytes and/or neurons. The methods are further described in greater detail below. Briefly, the methods may include seeding stem cells into the first well 70 and the second well 90. The method may also include placing a perfusion initiating amount of a liquid medium into a first well 70, a second well 90, and a third well 110. In one embodiment, the liquid medium does not include growth factors.

### EXAMPLES

The embodiments described herein will be further clarified by the following examples.

### Example 1

### Perfusion Microplate Assembly and Flow Characterization

Experimental Protocol.

Flow dynamics between connecting source, sample, and waste wells in a perfusion microplate were studied by monitoring fluorescent signals in the source, sample and waste wells. The effect of the dimensions of filter paper strips employed in the perfusion microplate was also studied using two different widths of filter paper strips, e.g. 810 μm and 320 μm.

The perfusion microplates employed in these studies were assembled by manually cutting pieces of filter paper strips, applying an adhesive to a well frame, curing the adhesive, attaching the filter paper strips onto the cured adhesive, and attaching a polystyrene film to the well frame. More specifically, the perfusion microplates employed in these studies were assembled by manually cutting pieces of filter paper strips from a 110 mm diameter filter paper (Catalog No. 1450-110, Whatman Inc., Piscataway, N.J.). The filter paper strips had the following dimensions: 1) 810 μm in width, 115 μm in thickness, and 3.5 mm in length (hereinafter “the 810 μm strip”); and 2) 320 μm in width, 115 μm in thickness, and 3.5 mm in length (hereinafter “the 320 μm strip”).

Next, a thin layer of a mixture of liquid biocompatible adhesive (Dow Corning® BIO-PSA 7-4301 Silicone Adhesive, Dow Corning Corporation, Midland, Mich.) and hexane (H292-4, Fisher Scientific, Pittsburgh, Pa.) in a 1:1 v/v % ratio was applied to the bottom of a standard flat bottom 96-well frame (Corning Incorporated, Corning, N.Y.). The hexane was employed to reduce the viscosity of the liquid biocompatible adhesive so that it could be applied easily and uniformly.

After applying the mixture of the liquid biocompatible adhesive and hexane, the microplate was cured at 60° C. for 1H in an oven. After curing, two filter paper strips were positioned and aligned between a group of three wells, and attached onto the cured adhesive surface under a microscope.

After attaching the filter paper strips onto the microplate, a layer of polystyrene film was adhered to the edges of the well bottom. Care was taken to ensure that the polystyrene film was fully adhered to the edges of the well bottom without any air bubbles. As shown in FIGS. 6A and 6B, each group of three wells was fluidly connected on the bottom of the wells by the filter paper strips once liquid was pipetted into the wells. Each individual filter paper strip connection was inspected under a microscope to confirm that no gaps were formed between the adhesive, the filter paper strips and the polystyrene film.

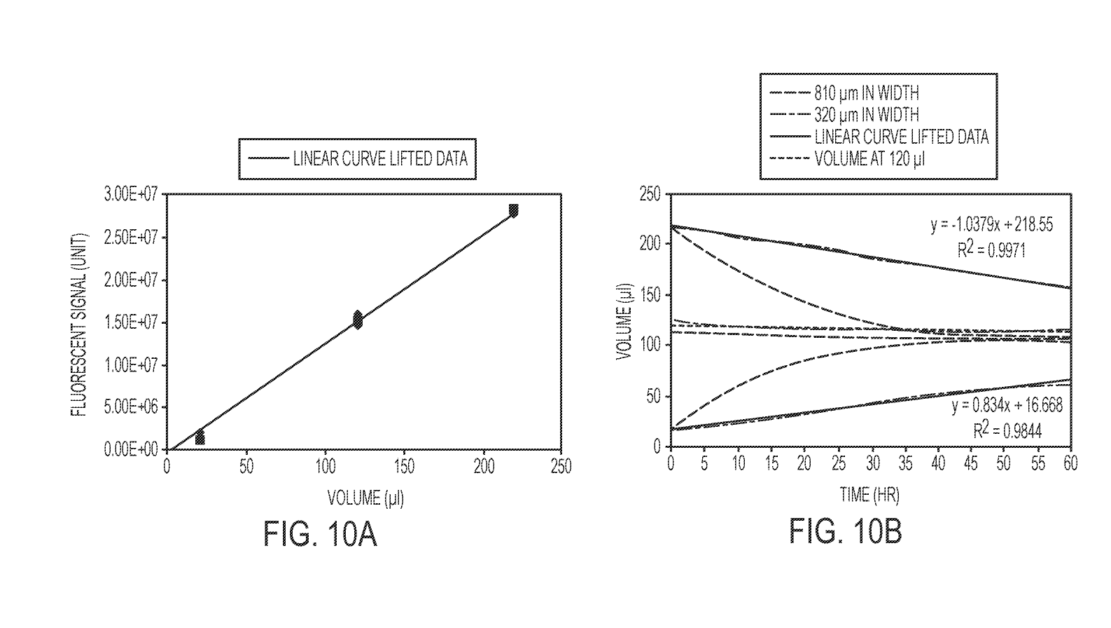

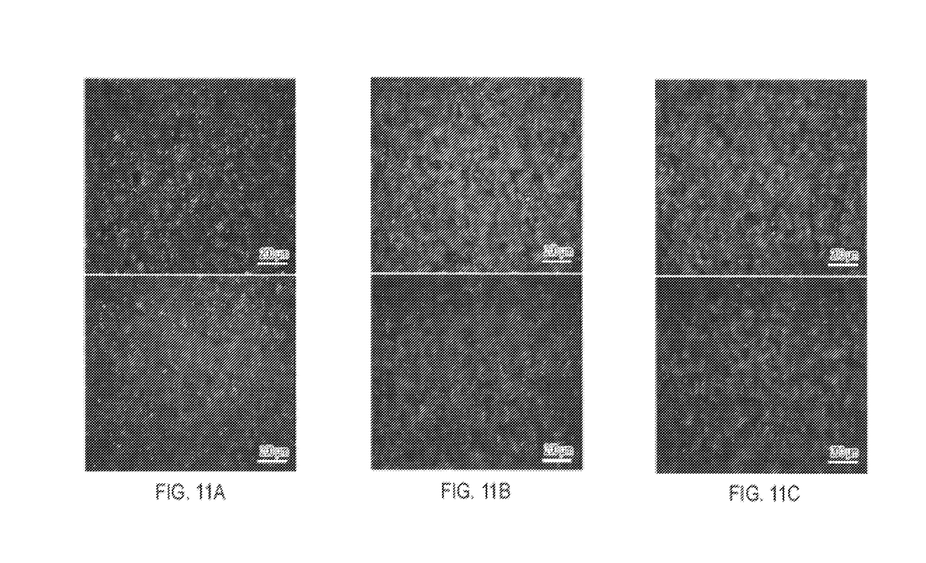

Flow dynamics between each group of three connecting wells (source, sample, and waste wells) were experimentally characterized using carboxyfluorescein fluorescent dye (8×10⁻⁵ M) (Catalog No. 54115, Sigma-Aldrich, St. Louis, Mo.) in phosphate buffered saline (hereinafter “PBS”) solution and a microplate reader (VICTOR3™ 1420 Multilabel Counter, PerkinElmer, Inc., Waltham, Mass.) at room temperature. Fluorescent dye solutions of 220 μl, 120 μl, and 20 μl were separately pipetted into the source, sample, and waste wells, respectively, and the fluorescent signal inside the wells was monitored in real-time by the microplate reader. As shown in FIG. 10A, in order to accurately monitor the liquid perfusion rates between connecting wells, a calibration curve of fluorescent signal as a function of fluorescent dye solution volume in the well of a conventional 96-well microplate was first obtained at room temperature using the microplate reader.

The effect of pore size of the perfusion membranes 130, 150 on liquid perfusion rates was also experimentally characterized as outlined above with regard to flow dynamics. However, in this study, cellulose membranes were employed as perfusion membranes. More specifically, cellulose strips having varying pore sizes of 1.2 μm, 5 μm, and 8 μm were employed as perfusion membranes. Additionally, fluorescent dye solutions of 220 μl, 120 μl, and 20 μl were separately pipetted into the source, sample, and waste wells. Fluorescent dye solutions of 290 μl, 50 μl, and 20 μl were separately pipetted into the source, sample, and waste wells.

Experimental Results.

As shown in FIG. 10B, automatic, continuous liquid perfusion between connecting wells was achieved by a combination of liquid wicking through the filter paper strips and the hydrostatic pressure between connecting wells. More specifically, automatic, continuous liquid perfusion between the source well and the sample well and between the sample well and the waste well was achieved by a combination of liquid wicking and hydrostatic pressure. Moreover, also as shown in FIG. 10B, because automatic, continuous liquid perfusion between connecting wells was achieved by the combination of liquid wicking through the filter paper strips and the hydrostatic pressure between connecting wells, it was discovered that liquid perfusion rates could be controlled by changing the dimensions of the filter paper strips. More specifically, as shown in FIG. 10B, linear perfusion rates were achieved between connecting wells by optimizing the filter paper strip dimensions at 320 μm in width, 115 μm in thickness, and 3.5 mm in length.

Additionally, liquid perfusion rates between connecting wells may also be controlled by optimizing the hydrostatic head, i.e. the liquid height differences between the source well and the sample well (h₁) or between the sample well and the waste well (h₂) (data not shown) and also by porosity (i.e., pore size) of the perfusion membranes 130, 150. In this study, in order to minimize the disturbance and fluctuation of the microenvironment in the sample well for better cell growth and yield, the same dimensions were employed for the filter paper strip connecting the source well and the sample well and for the filter paper strip connecting the sample well and the waste well. Additionally, the same hydrostatic heads were employed in between the source well and the sample well and in between the sample well and the waste well. As shown in FIG. 10B, such parameters resulted in the same liquid volume in the sample well throughout the perfusion period.

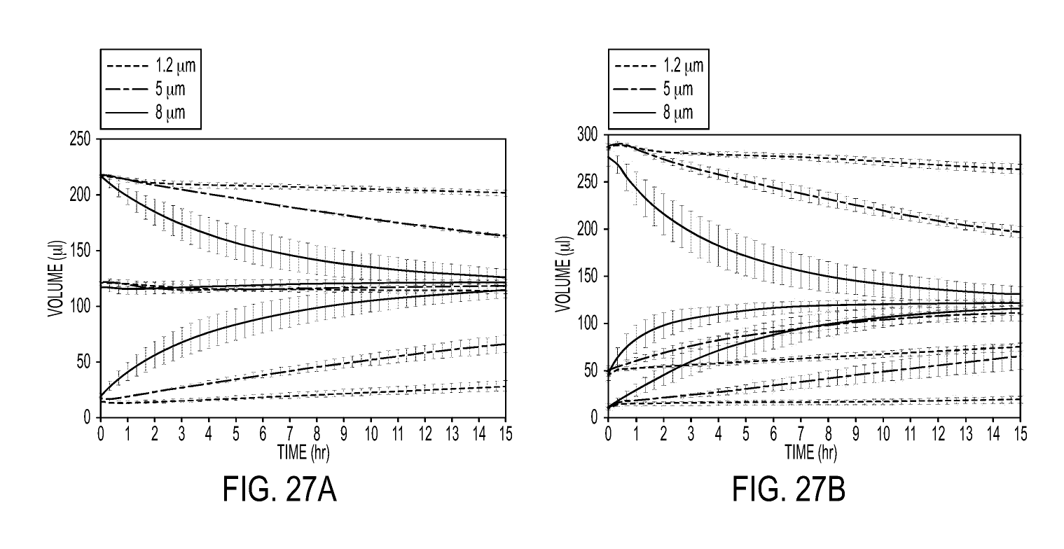

As shown in FIGS. 27A-B, liquid perfusion rates between connecting wells may also be controlled by adjusting the pore size of the perfusion membranes 130, 150.

### Example 2

### C3A Cell Culture

Experimental Protocol.

C3A cell viability of C3A cells cultured in conventional static microplates without medium exchange, cultured in conventional static microplates with medium exchange, and cultured in a perfusion microplate were studied. Perfusion microplates were prepared as previously described in Example 1, employing a filter paper strip with the dimensions 590 μm in width, 115 μm in thickness, and 3.5 mm in length (hereinafter “the 590 μm strip”).

With respect to C3A cell preparation, cryopreserved C3A cells, a derivative of HepG2/C3A human hepatoblastoma cell line (CRL-10741™, American Type Culture Collection, Manassas, Va.) were thawed and cultured in a sterile cell culture flask (Product #430641, Corning Incorporated, Corning, N.Y.) in Eagle's Minimum Essential Medium (hereinafter “EMEM”) (ATCC® No. 30-2003, ATCC, Manassas, Va.) supplemented with 10% fetal bovine serum (Catalog No. 16000-077, Invitrogen Corporation, Carlsbad, Calif.) and 1% Penicillin-Streptomycin (Catalog No. 15140-163, Invitrogen Corporation, Carlsbad, Calif.) in a CO₂ HEPA incubator (Model 3130 Form a Scientific, Inc., Marietta, Ohio) at 37° C., 95% humidity and 5% CO₂. Conventional static microplates and perfusion microplates were sterilized with 70% ethanol for 30 minutes, washed with deionized water, and air dried in a sterile cell culture hood. Next, C3A cells (50,000/well) from the cell culture flask were seeded into sample wells of the conventional static microplates and the perfusion microplates in 120 μl of Minimum Essential Medium (hereinafter “MEM”) (Catalog No. 41090-036, Invitrogen Corporation, Carlsbad, Calif.) and incubated at 37° C., 95% humidity and 5% CO₂.

For conventional static microplate-based cell culture experiments, C3A cells were cultured without medium exchange and with manual medium exchange. With regard to the conventional static microplate-based cell culture experiments with manual medium exchange, 100 μl of the 120 μl of MEM was exchanged daily. For the perfusion microplate-based cell culture experiments, no manual medium exchange was performed throughout the experiments and 220 μl, 120 μl, and 20 μl of MEM were respectively added to the source, sample, and waste wells.

After 4 days of cell culture, live and dead staining was performed to determine C3A cell viability (live and dead). The LIVE/DEAD® Viability/Cytotoxicity Assay Kit for mammalian cells (Molecular Probes, Inc., Eugene, Oreg.) was employed to determine viability of the cultured cells. Fluorescent dye mixture was pipetted into the cell culture wells followed by PBS buffer wash. Fluorescent live and dead staining images were collected using a Zeiss Axiovert 200 inverted fluorescence microscope equipped with an epifluorescence condenser and camera system (Carl Zeiss MicroImaging, Inc., Thornwood, N.Y.).

Experimental Results.

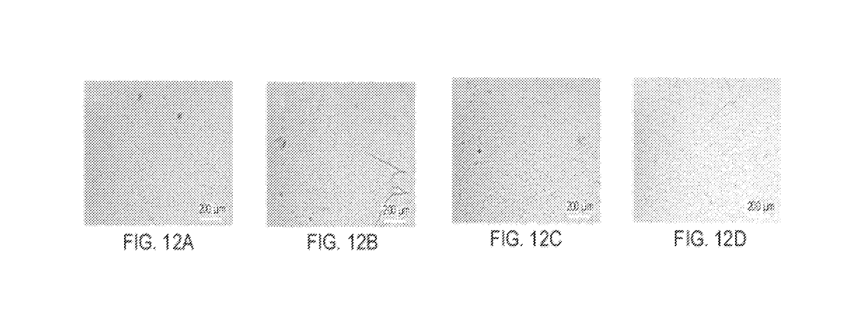

As shown in FIG. 11A, a large amount of C3A cells cultured in the conventional static microplate without medium exchange were dead after 4 days of cell culture. However, as shown in FIG. 11B, C3A cells cultured in the conventional static microplate with daily manual medium exchange were healthier with fewer dead cells after 4 days of cell culture. These results demonstrate the need for medium exchange to sustain healthy cell growth as shown in FIG. 11B.

As shown in FIG. 11C, live and dead staining of C3A cells cultured in the perfusion microplate (without manual medium exchange) revealed excellent cell growth with few dead cells after 4 days of cell culture. Moreover, comparing FIGS. 11B and 11C, C3A cells cultured in the perfusion microplate demonstrated comparable, if not better, cell growth compared to C3A cells cultured in the conventional static microplate with daily manual medium exchange. These results clearly demonstrate that the perfusion microplate provides a dynamic cell culture microenvironment similar to in vivo-like cell culture, eliminating the need for manual medium exchange in microplate-based cell cultures.

### Example 3

### Cell-Cell Communication Between LADMAC and EOC 20 Cells

Experimental Protocol.

Cell-cell communication between LADMAC and EOC 20 cells employing conventional static microplates and perfusion microplates was studied. Perfusion microplates were prepared as previously described in Example 1, employing the 590 μm strip.

With respect to LADMAC conditioned medium preparation, LADMAC cells are a transformed cell line derived by transfecting mouse bone marrow cells highly enriched for macrophage progenitors after transfection with human cellular myc-homologous DNA sequences in the pBR325 plasmid (pR myc) and secrete the human growth factor colony stimulation factor 1 (hereinafter “CSF-1”). CSF-1 is capable of supporting the in vitro proliferation of mouse bone marrow macrophages. The LADMAC cell line (CRL-2420™, ATCC, Manassas, Va.) is used to produce the CSF-1 containing LADMAC conditioned medium which will support the growth of the macrophage cell lines EOC 2 (CRL-2467™), EOC 13.31 (CRL 2468™), EOC 20 (CRL-2469™), I-11.15 (CRL-2470™) and I-13.35 (CRL-2471™). With respect to LADMAC cell preparation, cryopreserved LADMAC cells were thawed and cultured in a sterile cell culture flask in EMEM supplemented with 10% FBS at 37° C., 95% humidity and 5% CO₂. Medium was manually changed every 2 to 3 days.

LADMAC conditioned medium was prepared from LADMAC cells. Specifically, LADMAC cells were cultured to become confluent in the cell culture flask as previously described. After 5-7 days of cell culture, medium (supernatant) was collected from the cell culture flask and was centrifuged (Eppendorf® Centrifuge 5810 R, Eppendorf AG, Hamburg, Germany) at 125×g for 5 to 10 minutes. Next, the centrifuged medium was filtered using a 0.22 μm filter (Corning® 430767, Corning Incorporated, Corning, N.Y.). Finally, the filtered medium was stored at −20° C. for storage and for later use.

EOC 20 cells are an immortalized cell line derived from the brain of an apparently normal 10 day old mouse. EOC 20 cells depend on the growth factor CSF-1, secreted by LADMAC cells, for growth. With respect to EOC 20 cell preparation, cryopreserved EOC 20 cells (CRFL-2469™, ATCC, Manassas, Va.) were thawed and cultured in a sterile cell culture flask in Dulbecco's Modified Eagle's Medium (hereinafter “DMEM”) with 4 mM L-glutamine adjusted to contain 4.5 g/L glucose, 1.5 g/L sodium bicarbonate and 1.0 mM sodium pyruvate (ATCC® No. 30-2002, ATCC, Manassas, Va.) supplemented with 10% FBS and 20% CSF-1 containing LADMAC conditioned medium.

In conventional static microplates, EOC 20 cells were first seeded (40,000/well) in 120 μl of DMEM with 10% FBS and supplemented with or without CSF-1 containing 20% LADMAC conditioned medium and 100 μl out of the 120 μl of medium was exchanged daily with or without supplementing it with CSF-1 containing 20% LADMAC conditioned medium.

In perfusion microplates, sample wells were seeded with EOC 20 cells (40,000/well) in 120 μl of DMEM. Source wells of the perfusion microplate were filled either by 220 μl of DMEM or were seeded with LADMAC cells (20,000/well) in 220 μl of EMEM supplemented with 10% FBS. The waste wells of the perfusion microplate were filled with 20 μl of DMEM. Perfusion microplates were incubated at 37° C., 95% humidity and 5% CO₂ for 3 days without any user intervention. After 3 days of cell culture, live and dead staining was performed to determine EOC 20 cell viability as previously described in Example 2.

Results.

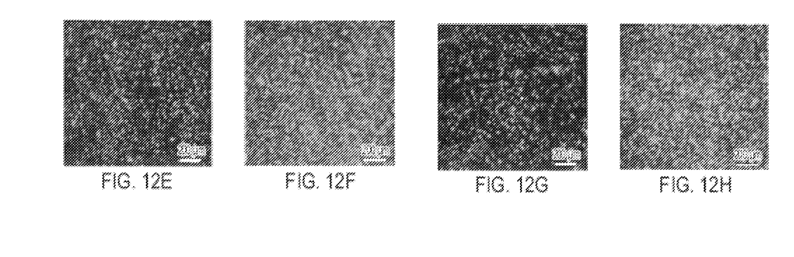

Comparing FIGS. 12A and 12E with 12B and 12F, EOC 20 cells cultured without supplementing the CSF-1 containing LADMAC conditioned medium in conventional static microplate wells exhibited poor cell growth with daily manual medium exchanges. Similarly, as shown in FIGS. 12C and 12G, EOC 20 cells cultured in the sample well without LADMAC cells cultured at the same time in the source well of the perfusion microplate also exhibited poor cell growth. However, as shown in FIGS. 12D and 12H, EOC 20 cells cultured in the sample well with LADMAC cells cultured at the same time in the source well of the perfusion microplate exhibited excellent growth. Additionally, comparing FIGS. 12D and 12H with 12B and 12F, such cell growth was comparable, if not better, to the daily CSF-1 containing LADMAC condition medium supplemented cell culture in the conventional static microplate wells. These results indicate that that CSF-1 secreted by the LADMAC cells was continuously perfused from the source well to the sample well in the perfusion microplate. As such experiments did not require the preparation of CSF-1 containing LADMAC conditioned medium (which typically takes around 5-7 days), the use of perfusion microplate saved time and costs associated with the manual labor in similar conditioned medium supplemented cell culture.

### Example 4

### Cross Configuration Perfusion Microplate Flow Characterization

Experimental Protocol.

Flow dynamics between connecting source (second), second source (fourth), sample (first), waste (third), and second waste (fifth) wells in a cross configuration perfusion microplate were studied by monitoring fluorescent signals in the source, second source, sample, waste, and second waste wells.

Perfusion microplates were prepared as previously described in Example 1, with distinctions provided in greater detail below. Instead of employing filter paper as a perfusion membrane, cellulose acetate perfusion membranes having a porosity of 1.2 μm (Catalog No. 1040312, Whatman, Inc., Piscataway, N.J.) were employed. The cellulose acetate perfusion membranes had the following dimensions: 1105 μm in width, 140 μm in thickness, and 3.5 mm in length (hereinafter “the 1105 μm membrane”). After curing, four cellulose acetate perfusion membranes were positioned and aligned between a group of five wells in a cross configuration, and attached onto the cured adhesive surface under a microscope. More specifically, the four cellulose acetate perfusion membranes were positioned and aligned between the following wells in a cross configuration: 1) a source (second) well and a sample (first) well; 2) a second source (fourth) well and the sample (first) well; 3) a waste (third) well and the sample (first) well; and 4) a second waste (fifth) well and the sample (first) well.

Flow dynamics between each group of five connecting wells (source, second source, sample, waste, and second waste wells) were experimentally characterized using carboxyfluorescein dye (4×10⁻⁵ M) in PBS solution and a microplate reader at room temperature as previously described in Example 1. Fluorescent dye solutions of 220 μl, 220 μl, 120 μl, 20 μl and 20 μl were separately pipetted into the source, second source, sample, waste, and second waste wells, respectively, and the fluorescent signal inside the wells was monitored in real-time by the microplate reader.

Experimental Results.

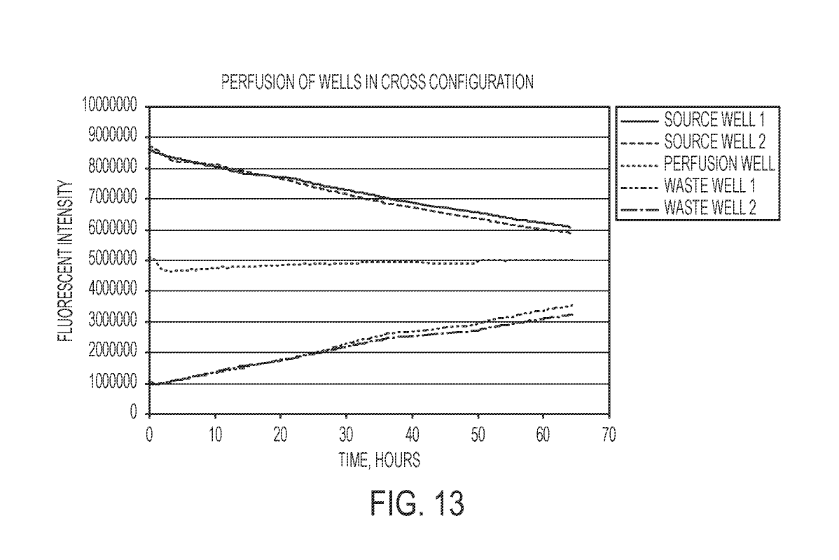

As shown in FIG. 13, automatic, continuous liquid perfusion between connecting wells was achieved by a combination of liquid wicking through the perfusion membrane and the hydrostatic pressure between connecting wells. More specifically, automatic, continuous liquid perfusion between the source well and the sample well, the second source well and the sample well, the waste well and the sample well, and the second waste well and the sample well was achieved by a combination of liquid wicking and hydrostatic pressure.

### Example 5

### Gradient Development in Sample Well in Cross Configuration Perfusion Microplate

Experimental Protocol.

Gradient development in a sample (first) well connected with a source (second), second source (fourth), waste (third), and second waste (fifth) well in a cross configuration perfusion microplate was studied by monitoring fluorescent signals of carboxyfluorescein and sulforhodamine B dyes across the sample well.

Cross configuration perfusion microplates were prepared as previously described in Example 4. In this experiment, the source (second), sample (first), and second source (fourth) wells were arranged in series, and the waste (third), sample (first), and second waste (fifth) wells were arranged in series. The two series intersected at the sample (first) well.

Gradient development in the sample well was experimentally characterized using carboxyfluorescein dye (4×10⁻⁵ M) in PBS solution, sulforhodamine B dye (8×10⁻⁵ M) (Invitrogen, Grand Island, N.Y.) in PBS solution and a Zeiss Axiovert 200 inverted fluorescence microscope (Carl Zeiss MicroImaging, Inc., Thornwood, N.Y.) equipped with standard fluorescein isothiocyanate and rhodamine filters (Omega Optical, Brattleboro, Vt.). 220 μl of the carboxyfluorescein dye in PBS solution was placed in the source (first) well. 220 μl of the sulforhodamine B dye in PBS solution was placed in the second source (fourth) well. 120 μl, 20 μl and 20 μl of PBS solution were respectively placed in the sample (first) well, the waste (third) well, and the second waste (fifth) well.

Flourescence intensity across the sample (first) well of sulforhodamine B dye in PBS solution and carboxyfluorescein dye in PBS solution was monitored at various time points (0 H, 0.5 H, 1 H, 1.5 H, 2 H, 2.5 H, and 3 H) with the Zeiss Axiovert 200 inverted fluorescence microscope. More specifically, fluorescence intensity was monitored across the sample (first) well from the inlet section of the source (first) well to the inlet section of the second source (fourth) well.

Experimental Results.

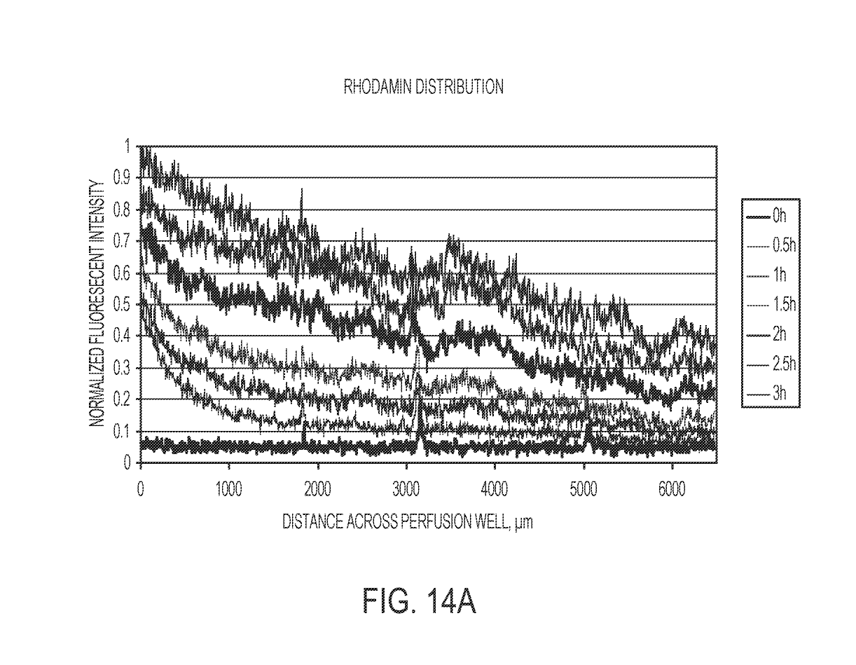

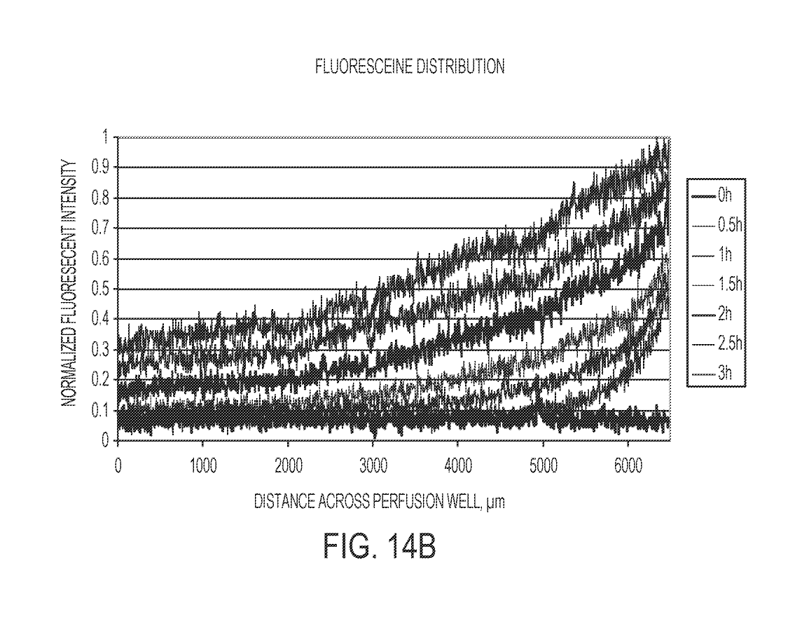

As shown in FIGS. 14A and 14B, a gradient developed across the sample (first) well upon initiating perfusion in a cross configuration perfusion microplate with sulforhodamine B dye in PBS solution and carboxyfluorescein dye in PBS solution.

### Example 6

### Toxicity Study of Primary Human Hepatocyte Metabolites of Prodrug Tegafur Toward HCT 116 Colon Cancer Cells

Experimental Protocol.

Toxicity of primary human hepatocyte metabolites of the chemotherapeutic prodrug Tegafur toward HCT 116 colon cancer cells in perfusion microplates was studied. The perfusion microplates employed in these studies were assembled by manually cutting pieces of cellulose acetate membrane strips, manually cutting channels in a pressure sensitive adhesive layer, applying the pressure sensitive adhesive layer to a polystyrene film, inserting the cellulose acetate membrane strips into the channels of the pressure sensitive adhesive layer, and attaching the polystyrene film to the well frame.

More specifically, the perfusion microplates employed in these studies were assembled by manually cutting pieces of cellulose acetate membrane strips from a 45 mm diameter cellulose acetate membrane paper (Catalog No. ST69, Whatman GmbH, Dassel, Germany). The cellulose acetate membrane strips had the following dimensions: 1) 1105 μm in width, 137 μm in thickness, and 3.5 mm in length. The cellulose acetate membrane strips had a pore size of 8 μm. Next, channels connecting three cavities in a double-sided pressure sensitive adhesive layer (ARcare 90106, Adhesive Research, Glen Rock, Pa.) were manually cut. The channels were 1200 μm wide. The three cavities in the double-sided pressure sensitive adhesive layer (hereinafter “PSA”) correspond in shape, size, and positioning to cavities of the well frame.

After manually cutting the channels, a first channel protective layer was removed from the double-sided pressure PSA and the unprotected side of the PSA was applied to the polystyrene film. After applying the PSA to the polystyrene film, two cellulose acetate membrane strips were positioned and aligned within the manually cut channels connecting three cavities in the double-sided PSA. Next, a second channel protective layer was removed from the double-sided pressure PSA and the unprotected side of the PSA was applied to a bottom surface of the well frame. Care was taken during such application of the PSA to the well frame to ensure that the three cavities in the double-sided PSA aligned with three cavities of the well frame, thereby forming a source, sample, and waste well. As shown in FIG. 9A, each group of source, sample, and waste wells were fluidly connected on the bottom of the wells by the cellulose acetate membrane strips once liquid was pipetted into the wells.

Primary human hepatocytes (50,000/well) (Invitrogen, Grand Island, N.Y.) were seeded in a source (second) well in 120 μl support medium F (hereinafter “MFE medium”) (Xenotech, Lenexa, Kans.) supplemented with 10% fetal bovine serum (hereinafter “FBS”) (Invitrogen, Grand Island, N.Y.). At approximately the same time, 120 μl buffer was respectively placed in the sample (first) well and the waste (third) well. The primary human hepatocytes were cultured for 1 day at 37° C. After culturing, a Matrigel™ (BD Biosciences, San Diego, Calif.) overlay was performed on the primary human hepatocytes in the source well. More specifically, the 120 μl MFE supplemented with 10% FBS was removed from the source well and was replaced with 120 μl 1:20 Matrigel™ dilution in serum free MFE medium. The primary human hepatocytes were cultured for 2 days at 37° C. in the 1:20 Matrigel™ dilution in the serum free MFE medium to establish polarity and restore phenotype specific functionality.

After culturing the primary human hepatocytes, the 120 μl buffer was removed from the sample (first) well and HCT 116 colon cancer cells (17,000/well) (Invitrogen, Grand Island, N.Y.) were seeded in the sample (first) well in 120 μl MFE medium supplemented with 10% FBS. The cells were cultured for 1 day at 37° C. After culturing, the 120 μl Matrigel™ dilution in serum free MFE medium was removed from the source well and was replaced with either 220 μl MFE medium, 220 μl MFE medium supplemented with Tegafur (40 μg/ml) (Sigma-Aldrich, St. Louis, Mo.), or 220 μl MFE medium supplemented with 5′-fluorouracil (hereinafter “5FU”) (Sigma-Aldrich, St. Louis, Mo.), to initiate automatic, continuous perfusion. Tegafur is a chemotherapeutic prodrug of 5FU which is metabolized by primary human hepatocytes. 5FU is used in the treatment of cancer. Additionally, at approximately the same time, 100 μl of the MFE medium supplemented with 10% FBS was removed from the waste (third) well. After 3 days of additional cell culture at 37° C., live and dead staining was performed to determine HCT 116 colon cancer cell viability as previously described in Example 2.

Experimental Results.

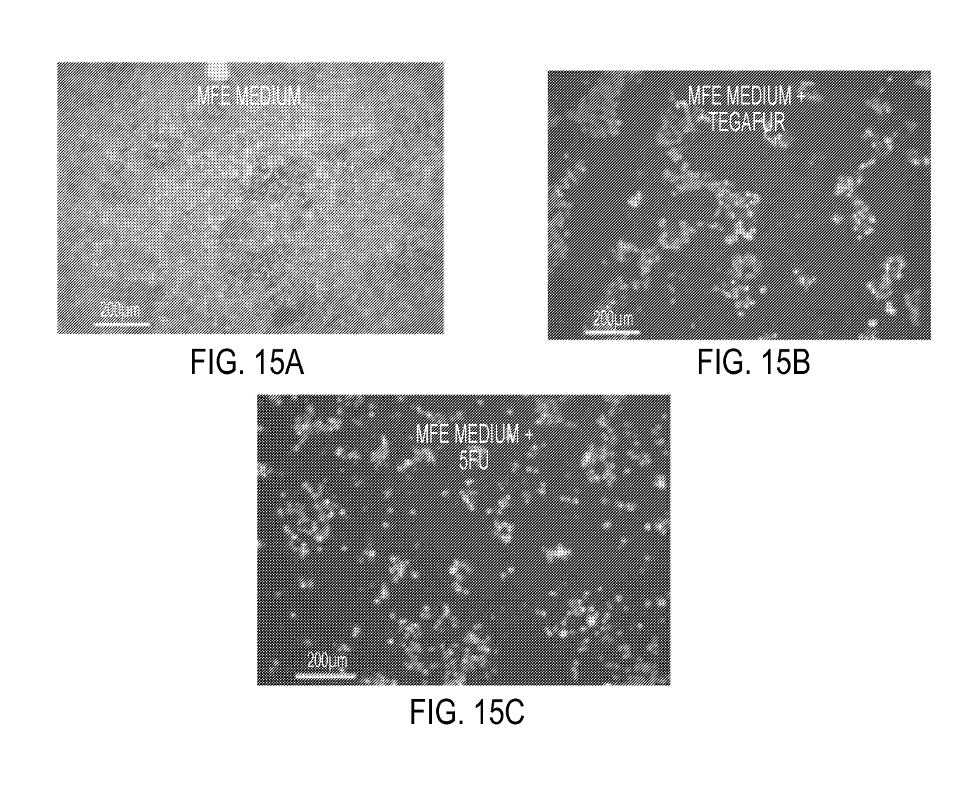

As shown in FIG. 15A, a large amount of HCT 116 colon cancer cells cultured with only MFE medium (without drug supplementation) were alive after 3 days of cell culture. However, as shown in FIGS. 15B and 15C, a large amount of HCT 116 colon cancer cells cultured respectively with MFE medium supplemented with Tegafur and MFE medium supplemented with 5FU, were dead after 3 days of cell culture. Moreover, comparing FIGS. 15B and 15C, MFE medium supplemented with Tegafur had a similar effect on HCT 116 colon cancer cells as MFE medium supplemented with 5FU. These results demonstrate the ability of the perfusion microplate to study the effects of drug metabolites.

### Example 7

### Toxicity Study of Prodrug Tegafur Toward HCT 116 Colon Cancer Cells without Primary Human Hepatocyte

Experimental Protocol.

Toxicity of the prodrug Tegafur toward HCT 116 colon cancer cells in perfusion microplates was studied. Perfusion microplates were prepared as previously described in Example 6.

HCT 116 colon cancer cells (17,000/well) were seeded in the sample (first) well in 120 μl MFE medium supplemented with 10% FBS. At approximately the same time, 120 μl MFE medium was placed in the source (second) well and 120 μl buffer was placed in the waste (third) well. The cells were cultured for 1 day at 37° C. After culturing, the MFE medium was removed from the source (second) well and was replaced with either 220 μl MFE medium, 220 μl MFE medium supplemented with Tegafur (40 μg/ml), or 220 μl MFE medium supplemented with 5′-fluorouracil (hereinafter “5FU”) to initiate automatic, continuous perfusion. Additionally, at approximately the same time, 100 μl of the buffer was removed from the waste (third) well. After 3 days of additional cell culture at 37° C., live and dead staining was performed to determine HCT 116 colon cancer cell viability as previously described in Example 2.

Experimental Results.

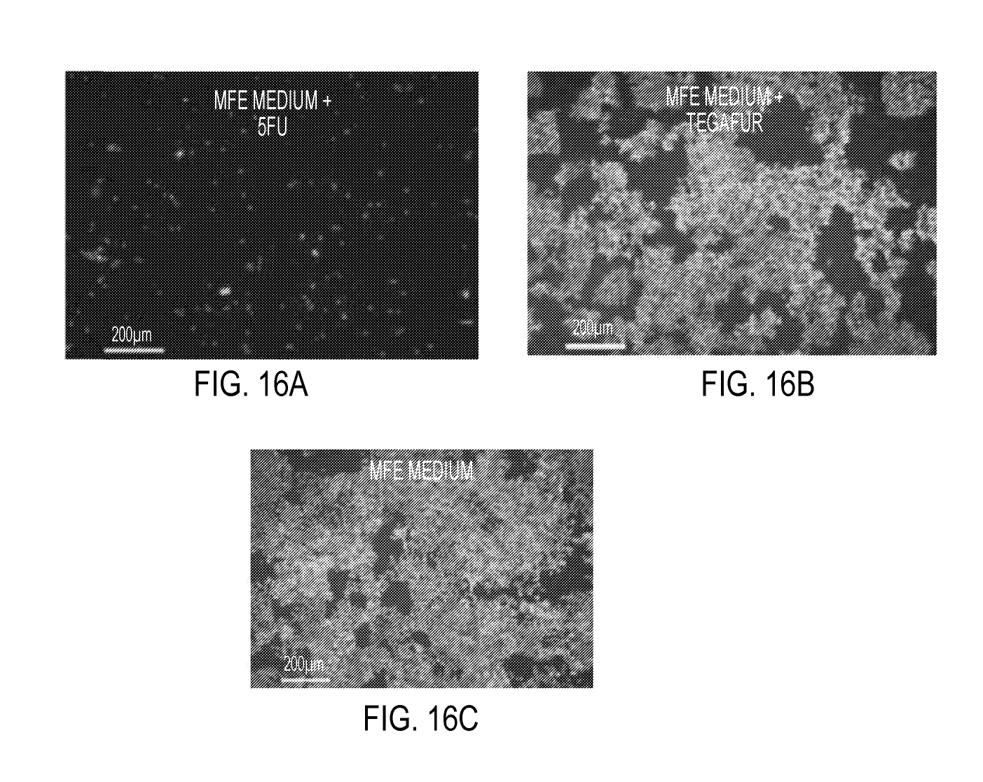

As shown in FIGS. 16B and 16C, a large amount of HCT 116 colon cancer cells cultured with either MFE medium supplemented with Tegafur or with only MFE medium (without drug supplementation) were alive after 3 days of cell culture. However, as shown in FIG. 16A, a large amount of HCT 116 colon cancer cells cultured respectively with MFE medium supplemented with 5FU were dead after 3 days of cell culture. Comparing FIGS. 15B and 16B, MFE medium supplemented with Tegafur in a source well seeded with primary human hepatocytes had a different effect on HCT 116 colon cancer cells in a sample well of a perfusion microplate than MFE medium supplemented with Tegafur without primary human hepatocytes in a source well on HCT 116 colon cancer cells in a sample well. These results further demonstrate the importance of the perfusion microplate in studying the effects of drug metabolites.

### Example 8

### Neural Progenitor Stem Cells Perfusion Culture

Experimental Protocol.

Viability, growth, and differentiation of human neural progenitor stem cells, ReNcell VM (Millipore, Billerica, Mass.), cultured in conventional static microplates without medium exchange, cultured in conventional static microplates with medium exchange, and cultured in a perfusion microplate, were studied. Perfusion microplates were prepared as previously described in Example 6, employing a cellulose acetate membrane with the dimensions 1200 μm in width, 137 μm in thickness, and 3.5 mm in length.

With respect to ReNcell VM preparation, ReNcell VM cells, (hereinafter “ReNcells”), were cultured on Laminin coated T75 tissue culture flasks (Corning Incorporated, Corning, N.Y.) in ReNcell NSC maintenance medium (Millipore, Billerica, Mass.) containing 20 ng/mL FGF-2 and 20 ng/mL EGF (Millipore). Medium change was performed every other day. Conventional static microplates and perfusion microplates were coated with Laminin. Next, ReNcells (5,000/well) were seeded into sample wells of the conventional static microplates, a Corning TCT microplate (Corning Incorporated, Corning, N.Y.), and the perfusion microplates.

In the perfusion microplate, an equal volume of medium was added to the source, sample, and waste wells to avoid any perfusion for the first 12 H. The day after seeding, perfusion in the perfusion microplate was started by simply respectively adjusting the volumes in each of the source, sample, and waste wells to 220 μl, 120 μl, and 20 μl of medium. The medium was perfused in the perfusion microplate continuously for 4 days.

In the conventional static microplate-based cell culture experiments, 120 μl of the medium was added to the sample wells. ReNcells were cultured without medium exchange and with manual medium exchange. With regard to the conventional static microplate-based cell culture experiments with manual medium exchange, 120 μl of the medium was changed every other day. With regard to the conventional static microplate-based cell cultures experiments without medium exchange, the medium was not changed for 4 days.

After 5 days of cell culture, colony morphology, expression of the stem cell marker Nestin, and cell growth were assessed using an Axiovert 200M inverted microscope (Zeiss, Thornwood, N.Y.). Expression of Nestin in ReNcells was assessed in conventional static microplates with medium exchange and in perfusion microplates via immunostaining with Nestin antibodies.

Experimental Results.

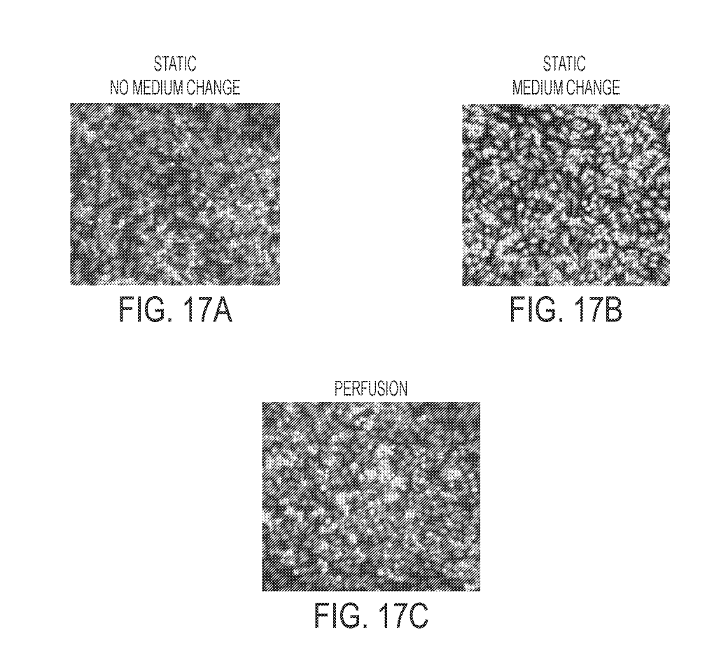

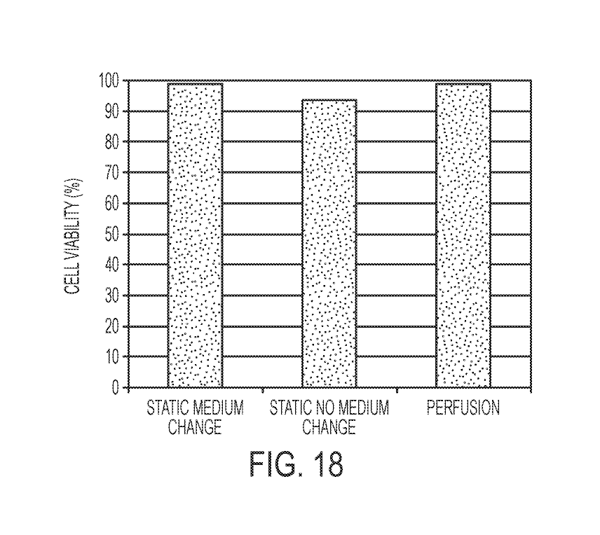

As shown in FIGS. 17A and 18, a large amount of ReNcells cultured in the conventional static microplate without medium exchange were dead after 4 days of cell culture. However, as shown in FIGS. 17B and 18, ReNcells cultured in the conventional static microplate with manual medium exchange were healthier with fewer dead cells after 4 days of cell culture. As shown in FIGS. 17C and 18, ReNcells cultured in the perfusion microplate revealed excellent cell viability with few dead cells after 4 days of cell culture. Comparing FIGS. 17B and 17C (and as shown in FIG. 18), ReNcells cultured in the perfusion microplate exhibited similar cell viability to ReNcells cultured in the conventional static microplate with medium exchange every two days. Additionally, comparing FIGS. 17A and 17C (and as shown in FIG. 18), cell viability of ReNcells cultured in the perfusion microplate was much better than ReNcells cultured in the conventional static microplate without medium exchange. These results demonstrate that the perfusion microplate provides a dynamic cell culture microenvironment for in-vivo-like cell culture.

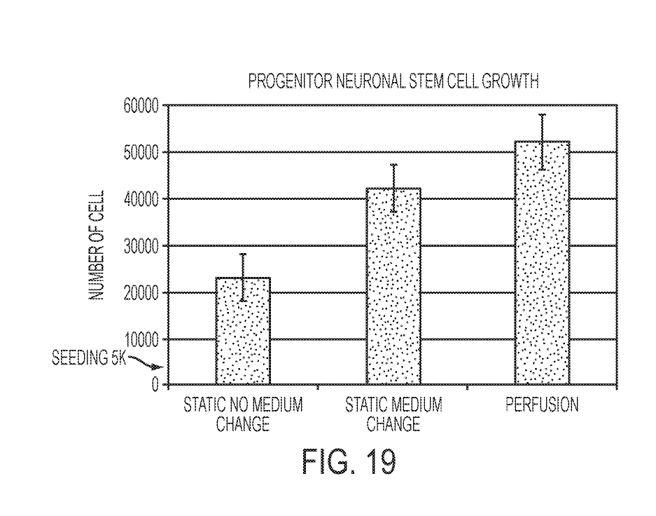

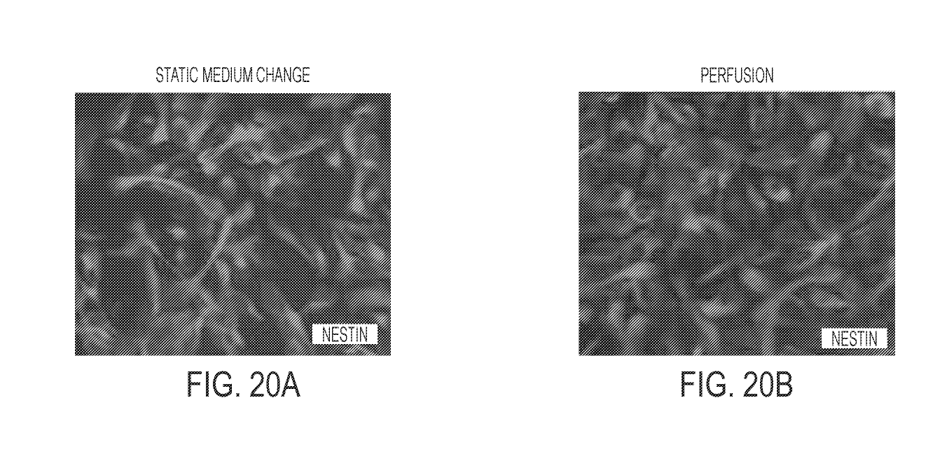

Additionally, as shown in FIG. 19, ReNcells cultured in the perfusion microplate exhibited better cell growth compared to both ReNcells cultured in the conventional static microplate with medium exchange and to ReNcells cultured in the conventional static microplate without medium exchange. Moreover, comparing FIGS. 20A and 20B, ReNcells cultured in the perfusion microplate remained undifferentiated (i.e., were Nestin positive) as compared to those cultured in the conventional static microplate with medium exchange.

### Example 9

### Perfusion Culture of hESCs and hiPSCs

Experimental Protocol.

Morphology, differentiation, and growth of hESCs and/or hiPSCs cultured in conventional static microplates without medium exchange, cultured in conventional static microplates with medium exchange, and cultured in a perfusion microplate, were studied. Among stem cells, hESCs and hiPSCs are known to have comparatively more specific culture requirements and to be comparatively more sensitive to environmental changes. Perfusion microplates were prepared as previously described in Example 6, employing a cellulose acetate membrane with the dimensions 1200 μm in width, 137 μm in thickness, and 3.5 mm in length. Cellulose acetate membranes had a pore size of 1.2 μm (#ST69, Whatman GmbH, Dassel, Germany), 5 μm (#AE99, Whatman GmbH), or 8 μm (#12342-7K, Sartorius Stedim Biotech GmbH, Goettingen, Germany).

With respect to hESC and hiPSC preparation, hESCs (BG01V/hOG cells, Invitrogen, Grand Island, N.Y.) or hiPSCs (Life Technologies, Grand Island, N.Y.) were maintained on Matrigel™ (BD Biosciences, San Diego, Calif.) coated Tissue Culture-Treated (i.e., TCT) 6 well plates (Corning Incorporated, Corning, N.Y.) in serum free mTERS1 medium (STEMCELL Technologies, Vancouver, BC) which was changed daily after the first 48H. Next, aggregated colonies were harvested by mechanical scraping and were resuspended in fresh mTERS1 medium. Cells (20,000/well) were then seeded into sample wells of the conventional static microplates, a Corning TCT 96 well microplate (Corning Incorporated, Corning, N.Y.), and the perfusion microplates, previously coated with Matrigel™

In the perfusion microplate, an equal volume of the medium was added to the source, sample, and waste wells to avoid any perfusion. Cells were allowed to attach to the Matrigel™ coated plates for 48H without any manipulation. Two days after seeding, perfusion in the perfusion microplate was started by respectively adjusting the volumes in each of the source, sample, and waste wells to 220 μl, 120 μl, and 20 μl of the medium. The medium was perfused in the perfusion microplate continuously for 4 days.

In the conventional static microplate-based cell culture experiments, 120 μl of the medium was added to the sample wells. Cells were cultured without medium exchange (serving as a negative control) and with manual medium exchange. With regard to the conventional static microplate-based cell culture experiments with manual medium exchange, 120 μl of the medium was changed daily.

After 5 days of cell culture, colony morphology, expression of the stem cell marker Oct-4, and cell growth were assessed using Axiovert 200M inverted microscope (Zeiss, Thornwood, N.Y.). Additionally, DAPI (i.e., 4′,6-diamidino-2-phenylindole) stains of hiPSCs and hESCs in perfusion 96-well microplates and in conventional static 96-well microplates with medium exchange were conducted. Expression of Oct-4 (i.e., octamer-binding transcription factor 4) in hiPSCs and hESCs in perfusion 96-well microplates and in conventional static 96-well microplates with medium exchange was also assessed via immunostaining with Oct-4 primary antibody.

Also in the perfusion microplate, the effect of varying liquid perfusion rates on cell growth of hESCs was assessed. More particularly, hESCs were cultured in sample wells of the perfusion microplates at a variety of liquid perfusion rates: 1 μL/H (i.e., FR1), 2 μL/H (i.e., FR2), and 20 1 μL/H (i.e., FR3). Cell growth was assessed by image analysis employing MetMorph 6.1 software.

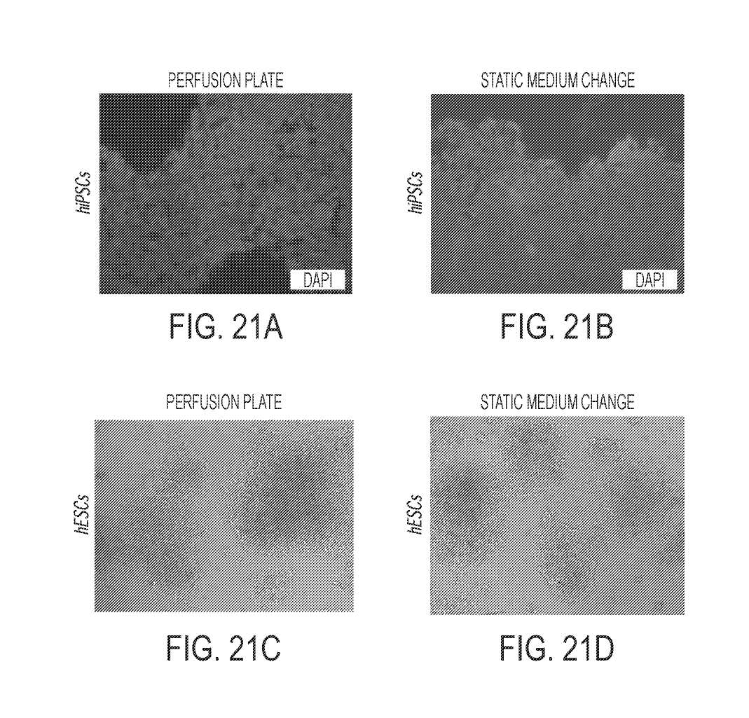

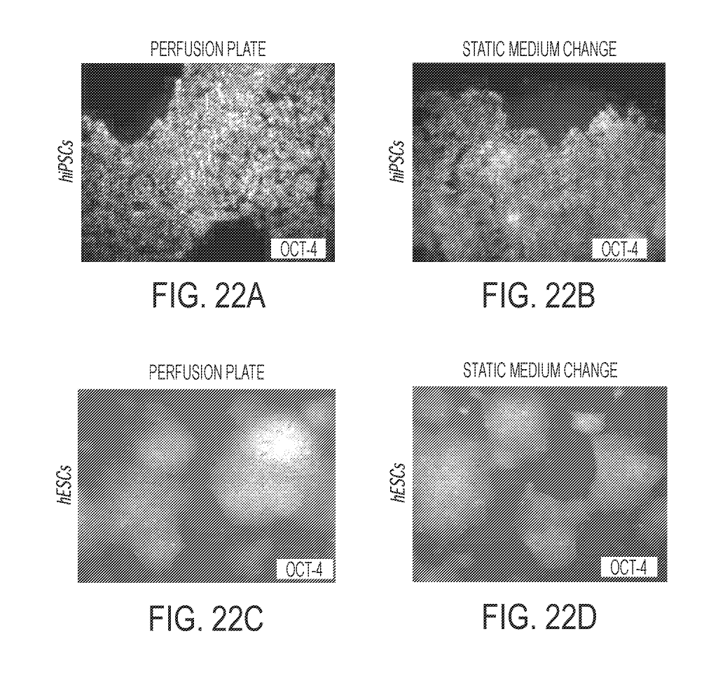

Experimental Results. As shown in FIGS. 21(A) and 22(A), hiPSCs cultured in perfusion microplates possess similar morphology and expression of the nuclear stem cell marker Oct-4 compared to hiPSCs cultured in conventional static microplates with medium exchange, such as is shown in FIGS. 21(B) and 22(B). Similarly, as shown in FIGS. 21(C) and 22(C), hESCs cultured in perfusion microplates possess similar morphology and expression of the nuclear stem cell marker Oct-4 compared to hESCs cultured in conventional static microplates with medium exchange, such as is shown in FIGS. 21(D) and 22(D).

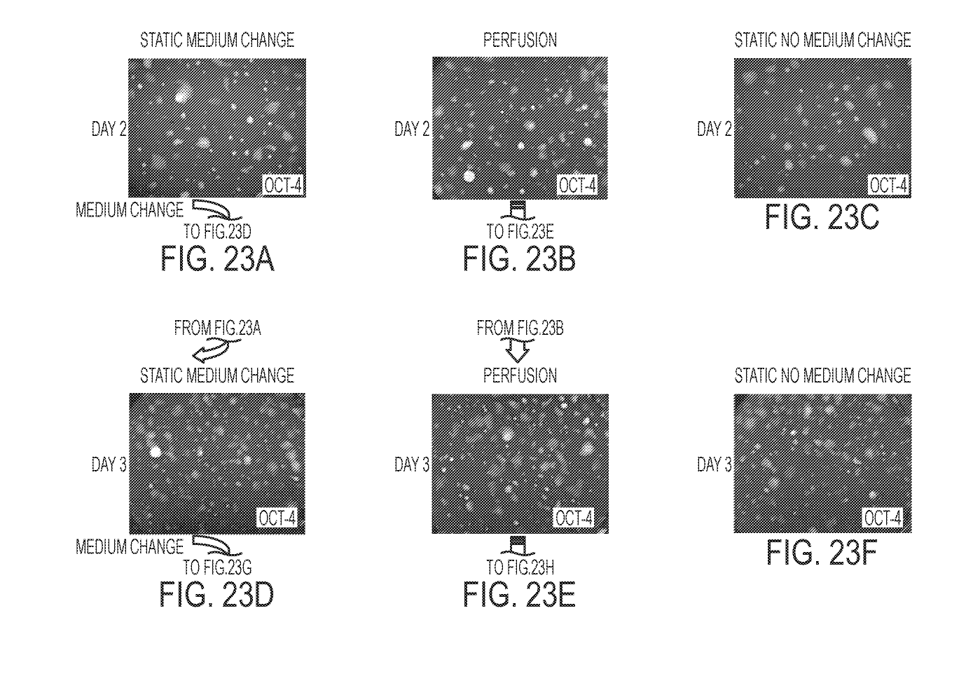

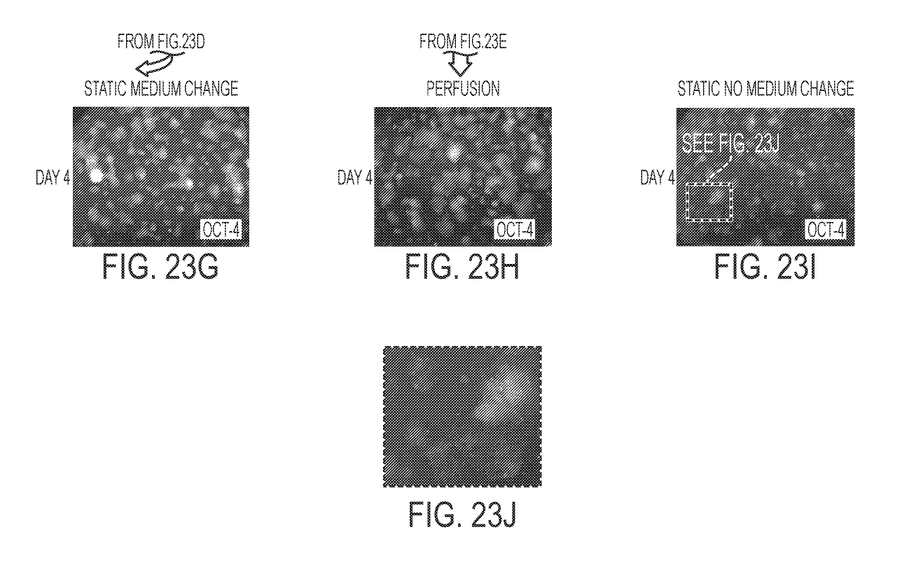

In contrast, as shown in FIGS. 23(C), 23(F), 23(I), and 23(J), hESCs cultured in conventional static microplates without medium exchange exhibited abnormal morphology and lost expression of the nuclear stem cell marker Oct-4 compared to hESCs cultured in conventional static microplates with medium exchange (shown in FIGS. 23(B), 23(E), and 23(H)), and compared to hESCs cultured in perfusion microplates (shown in FIGS. 23(A), 23(D), and 23(G)). Without being bound by the theory, such data indicates that daily medium exchange is necessary for the self-renewal characteristics of such cells.

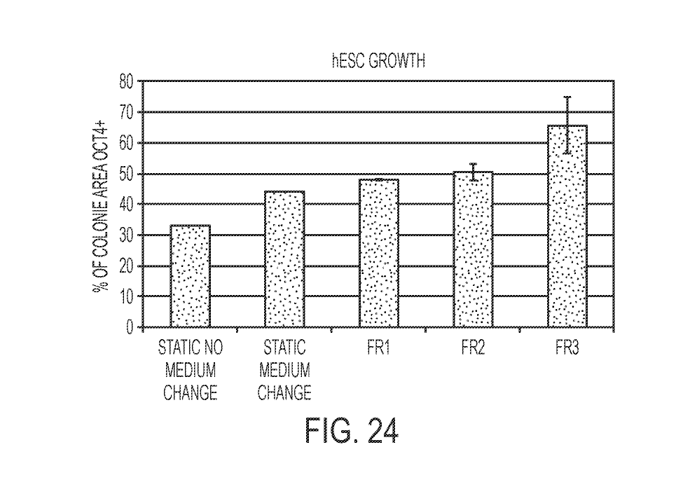

Finally, as shown in FIG. 24, hESCs cultured in sample wells of perfusion microplates with a liquid perfusion rate of 20 1 μL/H (i.e., FR3) exhibited better cell growth as compared to hESCs cultured in conventional static microplate wells without daily medium exchange and to hESCs cultured in conventional static microplates with daily medium exchange.

### Example 10

### Controlled Differentiation of ReNcells in Perfusion Culture

Experimental Protocol.

Controlled differentiation of ReNcells cultured in conventional static microplates with medium exchange and in a perfusion microplate was studied. Perfusion microplates were prepared as previously described in Example 6, employing a cellulose acetate membrane with the dimensions 1200 μm in width, 137 μm in thickness, and 3.5 mm in length. Such perfusion membrane has a porosity (i.e., pore size) of about 8 μm.

With respect to ReNcell preparation, ReNcells were cultured in Laminin coated T75 tissue culture flasks in ReNcell NSC maintenance medium containing 20 ng/mL FGF-2 and 20 ng/mL EGF. Differentiation of ReNcells was achieved by plating ReNcells (10,000/well) in the presence of growth factors into sample wells of the conventional static microplate, a Corning TCT microplate, and the perfusion microplate, previously coated with Laminin.

In the perfusion microplate, an equal volume of medium was added to the source, sample, and waste wells to avoid any perfusion for the first 12 H. The day after seeding, medium in the cell culture was exchanged with medium containing no growth factors. Perfusion in the perfusion microplate was started by simply respectively adjusting the volumes in each of the source, sample, and waste wells to 220 μl, 120 μl, and 20 μl of medium. The medium was perfused in the perfusion microplate continuously for 4 days. At the end of 4 days, 100 μL of fresh medium without growth factors was added to the source well and 100 μL of medium was removed from the waste well. The fresh medium was perfused in the perfusion microplate continuously for 3 days.

In the conventional static microplate-based cell culture experiments, 120 μl of the medium without growth factors was added to the sample wells. Medium was manually exchanged every other day.

After 8 days of culture, cell differentiation was assessed via immunostaining. Briefly, medium was removed from the sample wells and ReNcells were fixed for 15 minutes in cold 4% parafromaldehyde/PBS followed by two washes with PBS. ReNcells were permeabilized and blocked with 5% normal goat serum, i.e., NGS (Vector Labs, Burlingame, Calif.), and 0.3% TritonX-100 in PBS for 2 hours at room temperature. β-III Tubulin was probed using a mouse monoclonal at 1:1000 (Sigma, St. Louis, Mo.), and anti-GFAP rabbit polyclonal at 1:5000 (i.e., DAKO). Primary antibodies were incubated overnight at 4° C. After washing twice with PBS, cells were incubated with filtered Alexa dye conjugated Goat anti-Mouse 488 (1:250; Molecular Probes), or Alexa dye conjugated goat anti-rabbit 568 (1:2500; Molecular Probes) dissolved in 1% NGS in PBS for 1.5 H at room temperature. Cells were washed with PBS and counterstained with 10 mM Hoechst 33342 (Sigma) for 4 minutes followed by an additional washing with PBS.

Experimental Results.

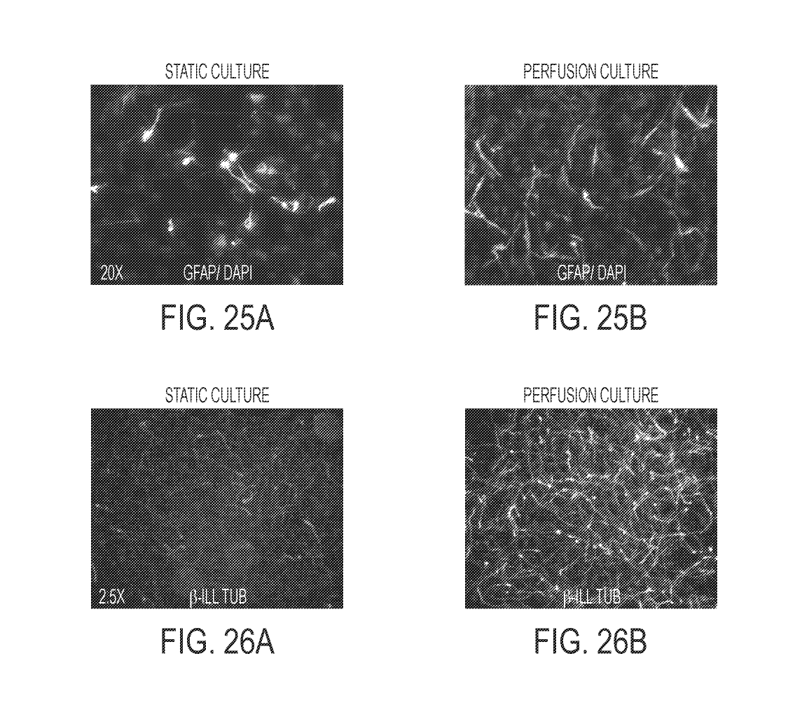

As respectively shown in FIGS. 25(A) and 25(B), progenitor neuronal stem cells differentiated into astrocytes in both ReNcells cultured in conventional static 96-well microplates with medium exchange and in ReNcells cultured in sample wells of perfusion microplates upon growth factor removal for 7 days. Such is exhibited by positive staining for GFAP marker. Additionally, as respectively shown in FIGS. 26(A) and 26(B), progenitor neuronal stem cells differentiated into neurons in both ReNcells cultured in conventional static 96-well microplates with medium exchange and in ReNcells cultured in sample wells of perfusion microplates upon growth factor removal for 7 days. Such is exhibited by β-III tubulin staining. However, significant improvement of differentiation was observed in ReNcells cultured in perfusion microplates as compared to ReNcells cultured in static 96-well microplates with medium exchange.

Comparing FIGS. 26 (A) with 26(B), it is clear that the number of neurons and the intensity of the fluorescence staining were dramatically increased in the ReNcells cultured in the perfusion microplate relative to ReNcells cultured in the static 96-well microplates. Moreover, ReNcells cultured in the perfusion microplates as compared to ReNcells cultured in static 96-well microplates with medium exchange appeared more mature. Cell morphology of differentiated neurons exhibited longer processes. Without being bound by the theory, such data demonstrates that ReNcells cultured with perfusion microplates improves the differentiation process and enables the production of more mature neuronal cells, which are critical hallmark parameters for predictive toxicity and drug discovery platforms. Additionally, because stem cell signaling and secretions may determine how a stem cell maintains its self-renewal property and/or specializes into various tissue types, without being bound by the theory, it is believed that the use of the perfusion microplate removes such secretions from stem cells before being transmitted to other cells. Accordingly, it is believed that the perfusion microplate may be employed to determine which exogenous factors may elicit specific cellular phenotypes.

It should now be understood that various aspects of the perfusion microplate are described herein and that such aspects may be utilized in conjunction with various other aspects.

In a first aspect, the disclosure provides a microplate for culturing cells with automatic, continuous perfusion of a liquid medium. The microplate includes a well frame which defines a plurality of cavities therethrough. The microplate further includes a planar substrate connected with the well frame. The planar substrate provides a bottom surface to the plurality of cavities, forming a plurality of wells. The plurality of wells includes a first well, a second well fluidly connected with the first well, and a third well fluidly connected with the first well. The first well is for culturing the cells in the liquid medium. The second well is for providing an outflow of the liquid medium to the first well. The third well is for receiving an inflow of the liquid medium from the first well. The second well is fluidly connected with the first well with a first perfusion membrane. The first perfusion membrane is disposed in between the well frame and the planar substrate and extends from an outlet section of the second well to an inlet section of the first well. The first perfusion membrane has a porosity range of from about 0.2 μm to about 200 μm. The third well is fluidly connected with the first well with a second perfusion membrane. The second perfusion membrane is disposed in between the well frame and the planar substrate and extends from an outlet section of the first well to an inlet section of the third well. The second perfusion membrane has a porosity range of from about 0.2 μm to about 200 μm. Upon introduction of a perfusion-initiating amount of the liquid medium into the second well, the liquid medium flows from the second well through the first perfusion membrane to the first well and from the first well through the second perfusion membrane to the third well.

In a second aspect, the disclosure provides a microplate of the first aspect, in which the first perfusion membrane and the second perfusion membrane have a porosity range of from about 0.2 μm to about 50 μm.

In a third aspect, the disclosure provides a microplate of the first or second aspect, in which each of the first perfusion membrane and the second perfusion membrane has a width of from about 0.2 mm to about 15 mm.

In a fourth aspect, the disclosure provides a microplate of any of the first to third aspects, in which each of the first perfusion membrane and the second perfusion membrane has a thickness of from about 50 μm to about 1000 μm and a length of from about 2 mm to about 55 mm.

In a fifth aspect, the disclosure provides a microplate of any of the first to fourth aspects, in which each of the first perfusion membrane and the second perfusion membrane has a width of about 1 mm.

In a sixth aspect, the disclosure provides a microplate of any of the first to fifth aspects, in which each of the first perfusion membrane and the second perfusion membrane independently formed of a hydrophilic material selected from the group consisting of: cellulose-derived polymers, nylons, polyethersulfones, polyamides, and cellulose filter papers.

In a seventh aspect, the disclosure provides a microplate of any of the first to the sixth aspects, in which each of the first perfusion membrane and the second perfusion membrane is formed of cellulose acetate.

In an eighth aspect, the disclosure provides a microplate of any of the first to the seventh aspects, in which the microplate also includes an adhesive layer disposed in between the well frame and the planar substrate which connects the well frame with the planar substrate, wherein the adhesive layer defines a plurality of cavities therethrough, and the plurality of cavities defined by the adhesive layer correspond in shape, size, and positioning with the plurality of cavities defined by the well frame.

In a ninth aspect, the disclosure provides a microplate according to the eighth aspect, in which the adhesive layer defines at least a first channel and a second channel, wherein the first channel corresponds in shape, size, and positioning with the first perfusion membrane, and wherein the second channel corresponds in shape, size, and positioning with the second perfusion membrane, such that the first perfusion membrane is disposed within the first channel and the second perfusion membrane is disposed within the second channel.

In a tenth aspect, the disclosure provides a microplate according to the eighth or the ninth aspect, in which the well frame defines at least a first groove and a second groove, wherein the first groove corresponds in shape, size, and positioning with the first perfusion membrane, and the second groove corresponds in shape, size, and positioning with the second perfusion membrane, such that the first perfusion membrane is disposed in the first groove and the second perfusion membrane is disposed in the second groove, and such that the first perfusion membrane and the second perfusion membrane are disposed in between the well frame and the planar substrate.

In an eleventh aspect, the disclosure provides a microplate according to any of the first to the tenth aspects, in which the plurality of wells further include a fourth well for providing an outflow of the liquid medium to the second well, wherein the fourth well is fluidly connected with the second well with a third perfusion membrane disposed in between the well frame and the planar substrate and extending from an outlet section of the fourth well to an inlet section of the second well, and the third perfusion membrane has a porosity range of from about 0.2 μm to about 200 μm, such that upon introduction of a perfusion-initiating amount of the liquid medium into the fourth well, the liquid medium flows from the fourth well through the third perfusion membrane to the second well, from the second well through the first perfusion membrane to the first well, and from the first well through the second perfusion membrane to the third well.

In a twelfth aspect, the disclosure provides a microplate according to any of the first to the tenth aspects, in which the plurality of wells further include a fourth well for receiving an inflow of the liquid medium from the third well, wherein the fourth well is fluidly connected with the third well with a third perfusion membrane disposed in between the well frame and the planar substrate and extending from an outlet section of the third well to an inlet section of the fourth well, and the third perfusion membrane has a porosity range of from about 0.2 μm to about 200 μm, such that upon introduction of a perfusion-initiating amount of the liquid medium into the second well, the liquid medium flows from the second well through the first perfusion membrane to the first well, from the first well through the second perfusion membrane to the third well, and from the third well through the third perfusion membrane to the fourth well.

In a thirteenth aspect, the disclosure provides a microplate according to the eleventh or the twelfth aspects, in which the first well is fluidly connected with the second well, the third well, and the fourth well in an inline configuration.

In a fourteenth aspect, the disclosure provides a microplate according to any of the first to the tenth aspects, in which the plurality of wells further includes a fourth well for providing a second outflow of the liquid medium to the first well and a fifth well for receiving a second inflow of the liquid medium from the first well, wherein the fourth well is fluidly connected with the first well with a third perfusion membrane disposed in between the well frame and the planar substrate and extending from an outlet section of the fourth well to a second inlet section of the first well, wherein the third perfusion membrane has a porosity range of from about 0.2 μm to about 200 μm, and the fifth well is fluidly connected with the first well with a fourth perfusion membrane disposed in between the well frame and the planar substrate and extending from a second outlet section of the first well to an inlet section of the fifth well, wherein the fourth perfusion membrane has a plurality of pores with diameters ranging from about 0.2 μm to about 200 μm.

In a fifteenth aspect, the disclosure provides a microplate according to the fourteenth aspect, in which the first well is fluidly connected with the second well, the third well, the fourth well, and the fifth well in a cross configuration.

In a sixteenth aspect, the disclosure provides a microplate according to any of the first to the fifteenth aspects, in which the well frame is formed from polymers and inorganic materials, or combinations thereof.

In a seventeenth aspect, the disclosure provides a microplate according to any of the first to the sixteenth aspects, in which the well frame is formed form a polymer selected from the group consisting of: hydrophilic polyethylene, polystyrenes, polypropylenes, acrylates, methacrylates, polycarbonates, polysulfones, polyesterketons, poly- or cyclic olefins, polychlorotrifluoroethylene, and polyethylene terephthalate.

In an eighteenth aspect, the disclosure provides a microplate according to any of the first to the sixteenth aspects, in which the well frame is formed from an inorganic material selected from the group consisting of: silicate, aluminosilicate, borosilicate, or boro-aluminosilicate, glass ceramics, ceramics, semiconductor materials, and crystalline materials.

In a nineteenth aspect, the disclosure provides a microplate according to any of the first to the seventeenth aspects, in which the well frame is formed from polystyrene.

In a twentieth aspect, the disclosure provides microplate according to any of the first to the sixteenth aspects, in which in which the planar substrate is formed from polymers and inorganic materials.

In a twenty-first aspect, the disclosure provides a microplate according to any of the first to the twentieth aspects, in which the planar substrate is connected with the well frame by a thermal weld, an infrared weld, or a chemical adhesive.

In a twenty-second aspect, the disclosure provides a microplate according to any of the first to the twenty-first aspects, in which the first well is fluidly connected with the second well and the third well in an inline configuration.

In a twenty-third aspect, the disclosure provides a microplate according to any of the first to the twenty-second aspects, in which the planar substrate includes a plurality of first and second areas, wherein the first areas provide the bottom surface to the plurality of cavities, and wherein the planar substrate is connected with the well frame at the plurality of second areas.

In a twenty-fourth aspect, the disclosure provides methods of fabricating a microplate according to any of the first to the twenty-third aspects.

In a twenty-fifth aspect, the disclosure provides methods of fabricating a microplate according to the twenty-fourth aspect, in which the method includes providing a well frame which defines a plurality of cavities therethrough, positioning at least one perfusion membrane on a bottom surface of the well frame such that each of the at least one perfusion membranes extends from a first cavity of the plurality of cavities to a second cavity of the plurality of cavities, wherein the second cavity is adjacent to the first cavity, wherein the at least one perfusion membrane has a porosity range of from about 0.2 μm to about 200 μm, and connecting a planar substrate with the well frame, wherein the planar substrate provides a bottom surface to the plurality of cavities thereby forming a plurality of wells, and wherein the at least one perfusion membrane is disposed in between the well frame and the planar substrate.

In a twenty-sixth aspect, the disclosure provides methods of fabricating a microplate according to the twenty-fourth or the twenty-fifth aspects, in which the planar substrate is connected with the well frame with a pressure sensitive adhesive.

In a twenty-seventh aspect, the disclosure provides methods of fabricating a microplate according to the twenty-sixth, in which the pressure sensitive adhesive is applied to the bottom surface of the well frame prior to the positioning of the at least one perfusion membrane on the bottom surface, such that the at least one perfusion membrane and the planar substrate adhere to the bottom surface of the well frame.

In a twenty-eighth aspect, the disclosure provides methods of fabricating a microplate according to any of the twenty-fourth to the twenty-seventh aspects, in which the method further includes forming at least one groove in the well frame prior to the positioning the at least one perfusion membrane on the bottom surface, wherein the at least one groove extends from the first cavity of the plurality of cavities to the second cavity of the plurality of cavities and corresponds in shape, size, and positioning with the at least one perfusion membrane such that when positioning the at least one perfusion membrane on the bottom surface of the well frame, the at least one perfusion membrane is positioned in the at least one groove.

In a twenty-ninth aspect, the disclosure provides methods of fabricating a microplate according to the twenty-sixth aspect, in which the pressure sensitive adhesive is an adhesive layer which is applied to the bottom surface of the well frame, and wherein the adhesive layer defines a plurality of cavities therethrough, and the plurality of cavities defined by the adhesive layer correspond in shape, size, and positioning with the plurality of cavities defined by the well frame, such that the planar substrate adheres to the adhesive layer and the at least one perfusion membrane is positioned in between the well frame and the planar substrate.

In a thirtieth aspect, the disclosure provides methods of fabricating a microplate according to any of the twenty-sixth aspect, in which the pressure sensitive adhesive is an adhesive layer which is applied to the bottom surface of the well frame, and wherein the adhesive layer defines a plurality of cavities therethrough, the adhesive layer defines at least one channel which extends from a first cavity of the plurality of cavities defined by the adhesive layer to a second cavity of the plurality of cavities defined by the adhesive layer and corresponds with the at least one perfusion membrane such that when positioning the at least one perfusion membrane on the bottom surface of the well frame, the at least one perfusion membrane is positioned within the at least one channel, and the plurality of cavities defined by the adhesive layer correspond in shape, size, and positioning with the plurality of cavities defined by the well frame, such that the planar substrate adheres to the adhesive layer and the at least one perfusion membrane is positioned in between the well frame and the planar substrate.

In a thirty-first aspect, the disclosure provides methods of culturing cells with automatic, continuous perfusion, in which the method includes providing a microplate according to any of the first to the twenty-third aspects, placing the cells to be cultured into at least one of the first well, the second well, and the third well of the microplate, and culturing the cells by placing a perfusion-initiating amount of the liquid medium into the second well through the first perfusion membrane to the first well and from the first well through the second perfusion membrane to the third well, contacting the cells.

In a thirty-second aspect, the disclosure provides methods of culturing cells according to the thirty-first aspect, in which the cells to be cultured are placed into the first well.

In a thirty-third aspect, the disclosure provides methods of culturing cells according to the thirty-first aspect, in which the cells to be cultured are placed into the first well and the second well.

In a thirty-fourth aspect, the disclosure provides methods of culturing cells according to the thirty-first aspect, in which the cells to be cultured are placed into the first well and the third well.

In a thirty-fifth aspect, the disclosure provides methods of culturing cells according to the thirty-first aspect, in which the cells to be cultured are placed into the first well, the second well, and the third well.

In a thirty-sixth aspect, the disclosure provides methods of culturing cells according to the thirty-third aspect, in which the method further includes performing an analysis of cell-cell communication between the cells cultured in the first well and the cells cultured in the second well, thereby determining an in vitro effect of the cell-cell communication on the cells cultured in the first well.

In a thirty-seventh aspect, the disclosure provides methods of culturing cells according to the thirty-sixth aspect, in which the analysis includes placing a perfusion initiating amount of a liquid medium into the second well such that the liquid medium contacts the cells cultured in the second well, flows from the second well through the first perfusion membrane to the first well, contacts the cells cultured in the first well, and flows from the first well through the second perfusion membrane to the third well.

In a thirty-eighth aspect, the disclosure provides methods of culturing cells according to the thirty-seventh aspect, in which the cells cultured in the first well include a first cell type and the cells cultured in the second well include a second cell type.

In a thirty-ninth aspect, the disclosure provides methods of culturing cells according to any of thirty-first to the thirty-fifth aspects, in which the method further includes performing an analysis of an effect of an agent of interest on the cultured cells, thereby determining an in vitro effect of the agent of interest on the cultured cells.

In a fortieth aspect, the disclosure provides methods of culturing cells according to the thirty-ninth aspect, in which the analysis includes placing a perfusion initiating amount of a liquid medium containing the agent of interest into the second well such that the agent of interest flows from the second well through the first perfusion membrane to the first well and from the first well through the second perfusion membrane to the third well, contacting the cultured cells and determining cell viability of the cultured cells.

In a forty-first aspect, the disclosure provides methods of culturing cells according to the thirty-third aspect, in which the method further includes performing an analysis of an effect of an agent of interest on the cells cultured in the first well, thereby determining an in vitro effect of the agent of interest on the cells cultured in the first well.

In a forty-second aspect, the disclosure provides methods of culturing cells according to the forty-first aspect, in which the analysis includes placing a perfusion initiating amount of a liquid medium containing the agent of interest into the second well such that the liquid medium contacts the cells cultured in the second well, flows from the second well through the first perfusion membrane to the first well, contacts the cells cultured in the first well, and flows from the first well through the second perfusion membrane to the third well; and determining cell viability of the cells cultured in the first well.

In a forty-third aspect, the disclosure provides methods of culturing cells according to the forty-second aspect, in which the agent of interest is a prodrug.

In a forty-fourth aspect, the disclosure provides methods of culturing cells according to the forty-third aspect, in which the agent of interest is a chemotherapeutic prodrug.

In a forty-fifth aspect, the disclosure provides methods of culturing cells according to the forty-third aspect, in which the cells cultured in the second well include a cell type capable of metabolizing the agent of interest.

It will be apparent to those skilled in the art that various modifications and variations can be made to the embodiments described herein without departing from the spirit and scope of the claimed subject matter. Thus it is intended that the specification cover the modifications and variations of the various embodiments described herein provided such modification and variations come within the scope of the appended claims and their equivalents.

What is claimed is:

1. A microplate for culturing cells with automatic, continuous perfusion of a liquid medium, the microplate comprising:
a well frame which defines a plurality of cavities therethrough; and
a planar substrate connected with the well frame, wherein the planar substrate provides a bottom surface to the plurality of cavities thereby forming a plurality of wells, wherein the plurality of wells comprise:
a first well for culturing the cells in the liquid medium;
a second well for providing an outflow of the liquid medium to the first well, wherein the second well is fluidly connected with the first well with a first perfusion membrane disposed in between the well frame and the planar substrate and extending from an outlet section of the second well to an inlet section of the first well, wherein the first perfusion membrane comprises a porosity range from about 0.2 μm to about 200 μm; and
a third well for receiving an inflow of the liquid medium from the first well, wherein the third well is fluidly connected with the first well with a second perfusion membrane disposed in between the well frame and the planar substrate and extending from an outlet section of the first well to an inlet section of the third well, wherein the second perfusion membrane comprises a porosity range from about 0.2 μm to about 200 μm, such that:
upon introduction of a perfusion-initiating amount of the liquid medium into the second well, the liquid medium flows from the second well through the first perfusion membrane to the first well and from the first well through the second perfusion membrane to the third well.

2. The microplate of claim 1, wherein each of the porosity range of the first perfusion membrane and the porosity range of the second perfusion membrane is from about 0.2 μm to about 50 μm.

3. The microplate of claim 1, wherein each of the first perfusion membrane and the second perfusion membrane comprises a width of from about 0.2 mm to about 15 mm, a thickness of from about 50 μm to about 1000 μm, and a length of from about 2 mm to about 55 mm.

4. The microplate of claim 1, wherein each of the first perfusion membrane and the second perfusion membrane comprises a width of about 1 mm.

5. The microplate of claim 1, wherein each of the first perfusion membrane and the second perfusion membrane independently comprises a hydrophilic material selected from the group consisting of: cellulose-derived polymers, nylons, polyethersulfones, polyamides, and cellulose filter papers.

6. The microplate of claim 5, wherein each of the first perfusion membrane and the second perfusion membrane comprises cellulose acetate.

7. The microplate of claim 1, further comprising an adhesive layer disposed in between the well frame and the planar substrate which connects the well frame with the planar substrate, wherein:
the adhesive layer defines a plurality of adhesive cavities therethrough; and
the plurality of cavities defined by the adhesive layer correspond in shape, size, and positioning with the plurality of cavities defined by the well frame.

8. The microplate of claim 7, wherein the adhesive layer defines at least a first channel and a second channel, wherein the first channel corresponds in shape, size, and positioning with the first perfusion membrane, and wherein the second channel corresponds in shape, size, and positioning with the second perfusion membrane, such that the first perfusion membrane is disposed within the first channel and the second perfusion membrane is disposed within the second channel.

9. The microplate of claim 7, wherein the well frame defines at least a first groove and a second groove, wherein the first groove corresponds in shape, size, and positioning with the first perfusion membrane, and the second groove corresponds in shape, size, and positioning with the second perfusion membrane, such that the first perfusion membrane is disposed in the first groove and the second perfusion membrane is disposed in the second groove, and such that the first perfusion membrane and the second perfusion membrane are disposed in between the well frame and the adhesive layer.

10. The microplate of claim 1, wherein the plurality of wells further comprise a fourth well for providing an outflow of the liquid medium to the second well, wherein:
the fourth well is fluidly connected with the second well with a third perfusion membrane disposed in between the well frame and the planar substrate and extending from an outlet section of the fourth well to an inlet section of the second well; and
the third perfusion membrane comprises a porosity range of from about 0.2 μm to about 200 μm, such that:
upon introduction of a perfusion-initiating amount of the liquid medium into the fourth well, the liquid medium flows from the fourth well through the third perfusion membrane to the second well, from the second well through the first perfusion membrane to the first well, and from the first well through the second perfusion membrane to the third well.

11. The microplate of claim 1, wherein the plurality of wells further comprise a fourth well for receiving an inflow of the liquid medium from the third well, wherein:
the fourth well is fluidly connected with the third well with a third perfusion membrane disposed in between the well frame and the planar substrate and extending from an outlet section of the third well to an inlet section of the fourth well, and
the third perfusion membrane comprises a porosity range of from about 0.2 μm to about 200 μm, such that:
upon introduction of a perfusion-initiating amount of the liquid medium into the second well, the liquid medium flows from the second well through the first perfusion membrane to the first well, from the first well through the second perfusion membrane to the third well, and from the third well through the third perfusion membrane to the fourth well.

12. The microplate of claim 11, wherein the first well is fluidly connected with the second well, the third well, and the fourth well in series.

13. The microplate of claim 1, wherein the plurality of wells further comprise a fourth well for providing a second outflow of the liquid medium to the first well and a fifth well for receiving a second inflow of the liquid medium from the first well, wherein:
the fourth well is fluidly connected with the first well with a third perfusion membrane disposed in between the well frame and the planar substrate and extending from an outlet section of the fourth well to a second inlet section of the first well, wherein the third perfusion membrane comprises a porosity range of from about 0.2 μm to about 200 μm, and
the fifth well is fluidly connected with the first well with a fourth perfusion membrane disposed in between the well frame and the planar substrate and extending from a second outlet section of the first well to an inlet section of the fifth well, wherein the fourth perfusion membrane comprises a plurality of pores with diameters ranging from about 0.2 μm to about 200 μm.

14. The microplate of claim 13, wherein the first well is fluidly connected with the second well, the third well, the fourth well, and the fifth well in a cross configuration.

15. A method of fabricating a microplate for culturing cells with automatic, continuous perfusion of a liquid medium, the method comprising:
providing a well frame which defines a plurality of cavities therethrough;
positioning at least one perfusion membrane on a bottom surface of the well frame such that each of the at least one perfusion membrane extends from a first cavity of the plurality of cavities to a second cavity of the plurality of cavities, wherein the at least one perfusion membrane comprises a porosity range of from about 0.2 μm to about 200 μm; and
connecting a planar substrate with the well frame, wherein the planar substrate provides a bottom surface to the plurality of cavities thereby forming a plurality of wells, and wherein the at least one perfusion membrane is disposed in between the well frame and the planar substrate.

16. The method of claim 15, wherein the planar substrate is connected with the well frame with a pressure sensitive adhesive.

17. The method of claim 16, wherein the pressure sensitive adhesive is applied to the bottom surface of the well frame prior to the positioning of the at least one perfusion membrane on the bottom surface, such that the at least one perfusion membrane and the planar substrate adhere to the bottom surface of the well frame.

18. The method of claim 16, further comprising forming at least one groove in the well frame prior to the positioning of the at least one perfusion membrane on the bottom surface, wherein the at least one groove extends from the first cavity of the plurality of cavities to the second cavity of the plurality of cavities and corresponds in shape, size, and positioning with the at least one perfusion membrane such that when positioning the at least one perfusion membrane on the bottom surface of the well frame, the at least one perfusion membrane is positioned in the at least one groove.

19. The method of claim 18, wherein the pressure sensitive adhesive comprises an adhesive layer which is applied to the bottom surface of the well frame, and wherein:
the adhesive layer defines a plurality of cavities therethrough; and
the plurality of cavities defined by the adhesive layer correspond in shape, size, and positioning with the plurality of cavities defined by the well frame, such that the planar substrate adheres to the adhesive layer and the at least one perfusion membrane is positioned in between the well frame and the adhesive layer.

20. The method of claim 16, wherein the pressure sensitive adhesive comprises an adhesive layer which is applied to the bottom surface of the well frame, and wherein:
the adhesive layer defines a plurality of cavities therethrough;
the adhesive layer defines at least one channel which extends from a first cavity of the plurality of cavities defined by the adhesive layer to a second cavity of the plurality of cavities defined by the adhesive layer and corresponds with the at least one perfusion membrane such that when positioning the at least one perfusion membrane on the bottom surface of the well frame, the at least one perfusion membrane is positioned within the at least one channel; and
the plurality of cavities defined by the adhesive layer correspond in shape, size, and positioning with the plurality of cavities defined by the well frame, such that the planar substrate adheres to the adhesive layer and the at least one perfusion membrane is positioned in between the well frame and the planar substrate.
